(19) United States
(12) Patent Application Publication
(10) Pub. No.: US 2024/0277907 A1
(43) Pub. Date: Aug. 22, 2024

(54) COMPOSITIONS OF EXTENDED RELEASE COATINGS AND METHODS FOR APPLYING EXTENDED RELEASE COATINGS

(71) Applicant: Eli Lilly and Company, Indianapolis, IN (US)

(72) Inventors: Christopher Luis BURCHAM, Zionsville, IN (US); Amy Leigh COX, Darlington, IN (US); Anthony David DUONG, Columbus, OH (US); Kaitlyn Mary EDDY, Norwood, MA (US); Jeffrey LeClair ELLIS, Columbus, OH (US); Eric Dwayne HAWKINS, Carmel, IN (US); Sarena Delvecchio HORAVA, Cambridge, MA (US); Thomas O. MERA, Grove City, OH (US); Coralie Adele RICHARD, Indianapolis, IN (US); Monica Rixman SWINNEY, Stoneham, MA (US)

(21) Appl. No.: 18/571,825

(22) PCT Filed: Jul. 22, 2022
(86) PCT No.: PCT/US2022/037998

§ 371 Date: Dec. 19, 2023

Related U.S. Application Data
(60) Provisional application No. 63/203,461, filed on Jul. 23, 2021.

(51) Int. Cl. A61L 29/16; A61L 29/04; A61L 29/08; A61L 29/12; A61M 5/142; A61M 25/00

(52) U.S. Cl. CPC A61L 29/16; A61L 29/041; A61L 29/085; A61L 29/126; A61M 5/14248; A61L 2300/41; A61L 2300/606; A61L 2420/02; A61L 2420/06; A61M 25/0009; A61M 25/0045

(57) ABSTRACT

A polymer coating including therapeutic agent particles for use with an extended wear infusion catheter is disclosed. Such a coating mitigates inflammation at the catheter site and provides introduction of the therapeutic agent at the infusion site for an extended time of at least 7-10 days to provide for continuous insertion of the catheter into a patient.

### FIELD OF THE DISCLOSURE

The present disclosure relates to a device for parenteral therapeutic agent delivery, more specifically methods and devices for continuous subcutaneous insulin infusion (CSII) configured for long term use with an extended therapeutic agent release coating.

### BACKGROUND

CSII may be performed using an insulin infusion set (IIS). One example of an IIS device 100 is shown in FIG. 1. The illustrative device 100 includes a first, proximal end 112 that communicates with an insulin reservoir of a pump (not shown) to receive an insulin formulation and a second, distal end 114 that communicates with a patient (not shown) to deliver the insulin formulation (i.e. the infusate). At the first end 112, the illustrative device 100 includes a reservoir connector 120 configured to couple with the insulin reservoir, a line set tubing 122, and a base connector 124. At the second end 114, the illustrative device 100 includes an infusion base 130 configured to receive the base connector 124, an adhesive pad 132 configured to adhere the infusion base 130 to the patient's skin, and an infusion catheter or needle 134 configured for insertion into the patient's skin. In use, the insulin formulation is directed from the pump, through the line set tubing 122, through the infusion catheter or needle 134, and into the patient's subcutaneous (SC) tissue.

IIS devices may vary in size, shape, appearance, materials, and other features. In one example, the material used to construct the infusion catheter 134 may vary (e.g., the Contact Detach™ Infusion Set available from Animas Corporation uses a steel infusion catheter, whereas the MiniMed® Quick-set® Infusion Set available from Medtronic uses a plastic infusion catheter). In another example, the arrangement of line set tubing 122 may vary (e.g., the Contact Detach™ Infusion Set available from Animas Corporation uses two sets of a line set tubing coupled together via an intermediate strain-relief base, whereas the MiniMed® Quick-set® Infusion Set available from Medtronic uses a single line set tubing).

The patient's body may exhibit an inflammatory and/or foreign body response at the site of the infusion catheter or needle 134. This response at the infusion site may vary from patient to patient depending on various factors, including the patient's susceptibility to wound formation, the patient's associated tissue remodeling and the patient's sensitivity to the particular insulin formulation, including to insulin analogs, for example, and to phenolic excipients (e.g., meta-cresol, phenol, methylparaben, ethylparaben, butylbaraben, other preservatives, and combinations thereof) in the insulin formulation, for example. M-cresol, in particular, has been shown to induce inflammatory pathways, negatively impact human immune cell types in vitro, degrade lipid bilayers and neuronal cell membranes, and induce aggregation of proteins and initiate protein unfolding which might contribute to infusion site events.

Known IIS devices for CSII are currently indicated for two-to three-day (2-3 d) use. After even a short wear time, the inflammatory and/or foreign body response may impair the efficacy of the patient's infusion site, thereby limiting insulin uptake, increasing the risk of hyperglycemia, and limiting viable infusion site longevity. The limited wear time for IIS devices represents a two-to seven-times discrepancy compared with the wear time for continuous glucose monitors (CGMs), thus introducing an obstacle to achieving a convenient, fully integrated CSII/CGM artificial pancreas system.

### SUMMARY

A polymer coating including therapeutic agent particles for use with an extended wear infusion catheter is disclosed. Such a coating mitigates inflammation at the catheter site and provides introduction of medication at the site for an extended time of at least fourteen days to provide for continuous insertion of the catheter into a patient.

According to an illustrative embodiment of the present disclosure, an infusion device is provided including a base, an adhesive configured to couple the base to a skin of a patient, and a catheter having a therapeutic coating. The coating includes a polymer matrix and a therapeutic agent. The coating defines a thickness of at least 40 μm (micrometers) and a therapeutic agent loading of at least 30 wt. % (weight percent).

According to another illustrative embodiment of the present disclosure, an extended release coating is provided including a polymer matrix having a therapeutic agent comprising at least 45 wt. % of the coating. The coating defines a thickness of at least 40 μm.

According to yet another illustrative embodiment of the present disclosure, a method for applying a therapeutic coating to a catheter is provided. The method includes compounding a mixture of a polymer and a therapeutic agent to form a polymer matrix and therapeutic agent mixture with a therapeutic agent loading of at least 30 wt. %. The method further includes shaping the mixture to form a coating for application to a catheter. The coating defines a thickness of at least 40 μm.

### BRIEF DESCRIPTION OF THE DRAWINGS

The above mentioned and other features of this present disclosure, and the manner of attaining them, will become more apparent and the invention itself will be better understood by reference to the following description of embodiments of the present disclosure taken in conjunction with the accompanying drawings, wherein:

FIG. 1 is a top plan view of a known insulin infusion set (IIS) device;

FIG. 2A is a cross-sectional view of an exemplary IIS device, the device including a reservoir connector, a line set tubing, a base connector, and an infusion base, wherein the device is applied to a patient's skin and includes a therapeutic coating on the infusion catheter;

FIG. 2B is a schematic cross-sectional view of a portion of the infusion catheter and the therapeutic coating of FIG. 2A;

FIG. 3A is an illustration of a first tube design of a molded polymer-therapeutic agent coating;

FIG. 3B is an illustration of a second tube design of a molded polymer-therapeutic agent coating;

FIG. 4A is a perspective view of an exemplary micro-injection molded catheter including a polymer-therapeutic agent coating;

FIG. 4B is a side perspective and cross-sectional view of the exemplary micro-injection molded catheter including a polymer-therapeutic agent coating;

FIG. 5A is a graph illustrating the average level of meloxicam plasma concentration in rats treated with exemplary solvent cast EVA coupons over a period of 14 days;

FIG. 5B is a graph illustrating the average normalized-to-dose level of meloxicam plasma concentration in the rats referenced in FIG. 5A;

FIG. 6 is a plurality of graphs illustrating exemplary effects of a thick coating of siliconization on the exemplary coupons of FIG. 5B;

FIG. 7 is a graph illustrating exemplary in vivo therapeutic agent release profiles of the exemplary coupons of FIG. 5A.

FIG. 8 illustrates an exemplary catheter assembly including a needle;

FIG. 9 illustrates a compression tester having a movable table and a chuck to assist with the dipping of the catheter of FIG. 8 in a solution;

FIG. 10 illustrates the exemplary catheter assembly of FIG. 8 being dipped into a vial containing a solution;

FIG. 11 illustrates the exemplary catheter assembly of FIG. 8 after being dipped into the solution, wherein the solution forms a coating on the catheter assembly;

FIG. 12A is a graph illustrating the stress at maximum load of coupons pre-elution according to an exemplary embodiment;

FIG. 12B is a graph illustrating the stress at maximum load of the exemplary coupons of FIG. 12A post-elution;

FIG. 12C is a graph illustrating the comparative change in stress at maximum load of the exemplary coupons of FIG. 12A pre-elution and post-elution;

FIG. 13A is a graph illustrating the strain at maximum load of the exemplary coupons of FIG. 12A pre-elution;

FIG. 13B is a graph illustrating the strain at maximum load of the exemplary coupons of FIG. 12A post-elution;

FIG. 13C is a graph illustrating the comparative change in strain at maximum load of the exemplary coupons of FIG. 12A pre-elution and post-elution;

FIG. 14A is a graph illustrating the Young's Modulus of the exemplary coupons of FIG. 12A pre-elution;

FIG. 14B is a graph illustrating the Young's Modulus of the exemplary coupons of FIG. 12A post-elution;

FIG. 14C is a graph illustrating the comparative change in Young's Modulus of the exemplary coupons of FIG. 12A pre-elution and post-elution;

FIG. 15A is a table illustrating the characteristics of a plurality of exemplary catheters and cross-sectional geometries of a subset of said catheters;

FIG. 15B illustrates the geometries of the exemplary catheters of FIG. 15A with dimensions reported in millimeter (mm);

FIG. 15C illustrates the results of mechanical testing of the catheters of FIG. 15A;

FIG. 16 is a graph illustrating the average therapeutic agent release profile of exemplary coupons having large therapeutic agent particles compared with the average therapeutic agent release profile of exemplary coupons having comparatively smaller therapeutic agent particles;

FIG. 17 is a graph illustrating the average therapeutic agent release profiles of prepared coated monofilaments, wherein the release profiles are normalized to the surface area of a 9 mm catheter;

FIG. 18 is a cross-sectional view of a coated monofilament (PCL-meloxicam coating), illustrating the concentric placement of the coating around the monofilament;

FIG. 19A is a graph illustrating the cumulative in vitro release profiles of coated monofilaments exemplified by FIG. 18, wherein the release profiles are normalized to the surface area of a 6 mm catheter;

FIG. 19B is a graph illustrating the cumulative in vitro release profiles of coated monofilaments exemplified by FIG. 18, wherein the release profiles are normalized to the surface area of a 9 mm catheter;

FIG. 19C is a graph illustrating the in vitro release rate profiles of coated monofilaments exemplified by FIG. 18, wherein the release rate profiles are normalized to the surface area of a 6 mm catheter;

FIG. 19D is a graph illustrating the in vitro release rate profiles of coated monofilaments exemplified by FIG. 18, wherein the release rate profiles are normalized to the surface area of a 9 mm catheter;

FIG. 20 illustrates exemplary molded cylinder parts having a first shot of HDPE for the 6 mm catheter length;

FIG. 21 illustrates exemplary two-shot cylinder parts having a first shot of HDPE and a second shot of PCL having 50 wt. % meloxicam loading for the 9 mm catheter length;

FIG. 22 is a graph illustrating in comparison the in vitro cumulative release profiles of two-shot cylinder molded parts exemplified by FIG. 21 and coated monofilaments exemplified by FIG. 18, wherein the release profiles are normalized to the surface area of a 9 mm catheter;

FIG. 23A is a side view of a coated monofilament, illustrating a semi-rough surface of the coated monofilament;

FIG. 23B is a side view of a two-shot cylinder molded part, illustrating a smooth surface of the two-shot cylinder molded part in comparison with FIG. 23A;

FIG. 24 is an illustration of a maximum liner thickness and a minimum liner thickness of coated tubing according to one exemplary embodiment;

FIG. 25 is a table providing the exemplary thickness of different formulations of EVA-therapeutic agent and corresponding overall catheter outer diameters according to minimum and maximum target release rates for each of the maximum liner thickness and minimum liner thickness of FIG. 24, wherein the provided thicknesses are normalized to the surface area of a 13 mm catheter;

FIG. 26 is a table providing the exemplary thickness of different formulations of EVA-therapeutic agent and corresponding overall catheter outer diameters according to minimum and maximum target release rates for each of the maximum liner thickness and minimum liner thickness of FIG. 24, wherein the provided thicknesses are normalized to the surface area of a 9 mm catheter;

FIG. 27 is a table providing the exemplary thickness of different formulations of EVA-therapeutic agent and corresponding overall catheter outer diameters according to minimum and maximum target release rates for each of the maximum liner thickness and minimum liner thickness of FIG. 24, wherein the provided thicknesses are normalized to the surface area of a 6 mm catheter;

FIG. 28 is a table providing the exemplary thickness of different formulations of PCL-therapeutic agent and corresponding overall catheter outer diameters according to minimum and maximum target release rates for each of the maximum liner thickness and minimum liner thickness of FIG. 24, wherein the provided thicknesses are normalized to the surface area of a 13 mm catheter;

FIG. 29 is a table providing the exemplary thickness of different formulations of PCL-therapeutic agent and corresponding overall catheter outer diameters according to minimum and maximum target release rates for each of the maximum liner thickness and minimum liner thickness of FIG. 24, wherein the provided thicknesses are normalized to the surface area of a 9 mm catheter; and

FIG. 30 is a table providing the exemplary thickness of different formulations of PCL-therapeutic agent and corresponding overall catheter outer diameters according to minimum and maximum target release rates for each of the maximum liner thickness and minimum liner thickness of FIG. 24, wherein the provided thicknesses are normalized to the surface area of a 6 mm catheter.

Corresponding reference characters indicate corresponding parts throughout the several views. Although the exemplification set out herein illustrates embodiments of the present disclosure, in several forms, the embodiments disclosed below are not intended to be exhaustive or to be construed as limiting the scope of the invention to the precise forms disclosed.

### DETAILED DESCRIPTION

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail, although it will be apparent to those skilled in the relevant art that some features that are not relevant to the present invention may not be shown for the sake of clarity.

### Device

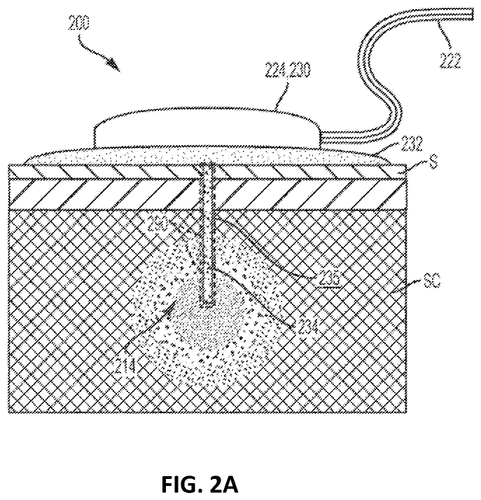

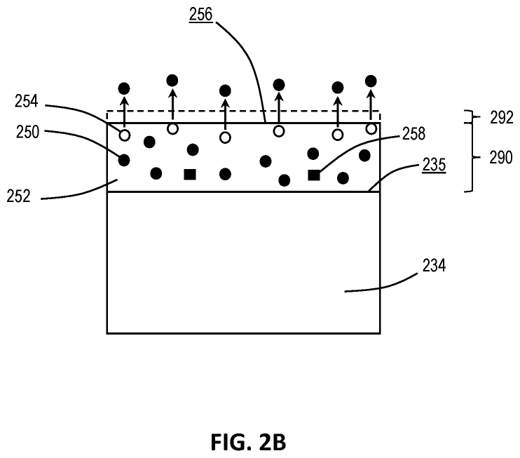

An exemplary IIS device 200 is shown in FIG. 2A for use with the therapeutic agent coatings and systems described herein. The device 200 includes a base connector 224 in the shape of a male buckle portion and an infusion set base 230 in the shape of a female buckle portion configured to receive the base connector 224. An adhesive pad 232 is configured to adhere the infusion base 230 and the coupled base connector 224 to the patient's skin S. An infusion element in the form of an infusion catheter 234 is configured for insertion into the patient's subcutaneous SC tissue and is fluidly coupled to the infusion base 230 and the base connector 224 of the device. It is also within the scope of the present disclosure for the infusion element to be a needle. Flexible line set tubing 222 fluidly couples the infusion base 230 and the base connector 224 to a reservoir connector (not shown) that is configured to couple with an insulin reservoir (not shown). In use, the insulin formulation is directed from the pump, through the line set tubing 222, through the fluidic path in the infusion set base 230, then through the infusion catheter 234, and into the patient's subcutaneous SC tissue.

The infusion catheter 234 may be constructed of steel, plastic (e.g., polytetrafluoroethylene (PTFE), fluorinated ethylene propylene (FEP), poly(propylene), high-density polyethylene (HDPE), low-density polyethylene (LDPE), ethyl vinyl acetate (EVA), copolymers thereof, and combinations thereof), or another suitable material. The infusion catheter 234 may be sufficiently thick to withstand implantation while being sufficiently thin to promote patient comfort. In some embodiments, the infusion catheter 234 may have a thickness less than about 200 μm, less than about 150 μm, or less than about 100 μm, for example. In some embodiments, the infusion catheter 234 may have an outer diameter of less than about 1 mm. In some embodiments, the infusion catheter 234 may have an outer diameter of about 0.7 mm to 0.8 mm.

A therapeutic coating 290 may be located on and/or in the device 200. The therapeutic coating 290 may be configured to release and deliver one or more therapeutic agents to the patient in an extended manner, as described further below.

The application of the therapeutic coating 290 to device 200 may vary. In certain embodiments, the therapeutic coating 290 may be incorporated (e.g., embedded) directly into device 200. In other embodiments, the therapeutic coating 290 may be applied (e.g., coated) onto an underlying surface of the device 200. In other embodiments, the therapeutic coating 290 may be applied onto a filtration mechanism that is loaded into the device 200.

The location of the therapeutic coating 290 on the device 200 may also vary. In the illustrated embodiment of FIGS. 2A and 2B, for example, the therapeutic coating 290 is coated onto an outer surface 235 of the infusion catheter 234 to substantially cover the outer surface 235. The therapeutic coating 290 may directly contact and then disperse into the patient's SC tissue along with the insulin formulation traveling through the device 200, which may reduce the magnitude or speed of the patient's inflammatory response. In other embodiments, and as noted above, the therapeutic coating 290 may be incorporated into the infusion catheter 234. In this embodiment, the infusion catheter 234 and the therapeutic coating 290 may be integrally formed of the same material.

It is also within the scope of the present disclosure for the therapeutic coating 290 to be located along the fluid pathway of the device 200. More specifically, the therapeutic coating 290 may be located inside the line set tubing 222, inside the base connector 224, inside the infusion base 230, and/or inside the infusion catheter 234 such that the therapeutic coating 290 may dissolve into the insulin formulation traveling through device 200 for simultaneous delivery to the patient.

In the embodiments described herein, the infusion site may last at least 3 days, 5 days, 7 days, 10 days, or more, such as about 7 to 14 days or up to 21 days, which may reduce insulin waste, improve glycemic control, reduce scarring, and enable a once-weekly or once-biweekly change-over time frame for a fully integrated artificial pancreas system. The device 200 may include various other features designed to achieve longevity in CSII infusion site viability. Further features and descriptions of features may be found in U.S. Patent Application Publication No. 2019/0054233 to DEMARIA, et al., published Feb. 21, 2019, and titled “INFUSION SET WITH COMPONENTS COMPRISING A POLYMERIC SORBENT TO REDUCE THE CONCENTRATION OF M-CRESOL IN INSULIN”, the disclosure of which is hereby expressly incorporated by reference in its entirety.

### Coating/Formulation

An exemplary therapeutic coating 290 is shown in more detail in FIG. 2B. As discussed above, the extended release therapeutic coating 290 may be applied to an outer surface 235 of the infusion catheter 234 of the exemplary device 200, which disperses one or more therapeutic agents at a controlled rate of release to locally treat the patient's body at the device site.

The therapeutic coating 290 may include one or more first therapeutic agents 250 in the form of anti-inflammatory agents, including nonsteroidal anti-inflammatory therapeutic agents (NSAIDs). Exemplary anti-inflammatory agents include meloxicam, bromfenac, ibuprofen, naproxen, aspirin, plumbagin, plumericin, celecoxib, diclofenac, etodolac, indomethacin, ketoprofen, ketorolac, nabumetone, oxaprozin, piroxicam, salsalate, sulindac, tolmetin, rapamycin, dexamethasone, betamethasone, heparin, sirolimus, and paxlitaxel, for example. A device site, and its corresponding device 200, may last longer when a NSAID is locally administered, resulting in further benefits for the patient, including use of fewer devices, fewer needle sticks, and avoidance of hyperglycemia that is associated with an inflammatory response. Controlled release of a NSAID from the outer surface 235 of the infusion catheter 234 locally at the insertion site may allow the device site and its corresponding device 200 to last for an extended time period longer than 3 days, 5 days, 7 days, 10 days, or more, such as about 7 to 14 days or up to 21 days.

The therapeutic coating 290 may also include other or secondary therapeutic agents 252 alone or in combination with the anti-inflammatory, first therapeutic agents 250. Exemplary secondary therapeutic agents 252 include inhibitors of tyrosine kinase (e.g., masitinib), inhibitors of the matri-cellular protein Thrombospondin 2 (TSP2), inhibitors of fibrosis-stimulating cytokines including Connective Tissue Growth Factor (CTGF), inhibitors of members of the integrin family of receptors, Vascular Endothelial Growth Factor (VEGF), local insulin receptor inhibitors, antimicrobial agents (e.g., silver) and diffusion enhancing agents (e.g., hyaluronidase), for example. In one particular example, the therapeutic coating 290 includes the secondary therapeutic agent VEGF in combination with the anti-inflammatory agent dexamethasone, but other combinations are also contemplated.

The therapeutic coating 290 may also include one or more polymers to form a matrix 252 for the therapeutic agent(s), which may improve film or coating properties, improve solubility or elution properties, and/or impart a time-release effect to elution of the therapeutic coating 290 into the patient's SC tissue. Exemplary polymers include ethyl vinyl acetate (EVA), polyethylene glycol (PEG), polyvinyl alcohol (PVA), polylactic acid (PLA), polyglycolic acid (PGA), poly(lactic-co-glycolic acid) (PLGA), polyhydroxyethylmethacrylate (PHEMA), poly(methacrylic acid) (PMAA), alginate (poly) phosphoryl chlorines and (poly) ester amides, polycaprolactone (PCL), thermoplastic polyurethane (TPU), hydroxypropyl methylcellulose (HPMC), co-povidone, copolymers thereof, and other combinations thereof, for example. The polymer matrix 252 may be a non-degradable material that remains substantially intact as the therapeutic agent 250, 258 diffuses from the polymer matrix 252 as described further herein.

Illustratively, as shown in FIG. 2B, the therapeutic coating 290 may include the first therapeutic agent 250, such as a NSAID (e.g., meloxicam) captured within the polymer matrix 252 (e.g., EVA, PCL, TPU). Specifically, particles of the NSAID 250 are discretely dispersed, rather than amorphously dispersed, within the polymer matrix as a crystalline solid dispersion 252. As discussed further herein, in other embodiments, an amorphous solid dispersion of a therapeutic agent within a polymer is formed, resulting in amorphous solid dispersion of the therapeutic agent. However, in the present embodiment, as illustrated by the Examples provided below, desirable release profiles of the therapeutic agent 250 are achieved by leaving the therapeutic agent 250 in its crystalline form. The crystalline therapeutic agents 250 also create discrete therapeutic agent particle domains that, after elution, leave behind pores 254 which allow bodily fluids to travel beneath the surface 256 of the polymer matrix 252 such that a greater amount of the therapeutic agent 250 is dissolved in the fluid and diffuses from further beneath the surface 256 of the polymer matrix 252 in an extended manner. Such discrete particles may be achieved by maintaining a compounding temperature above the melting point of the polymer 252, but below the melting point of the therapeutic agent 250 when melt compounding the polymeric therapeutic coating 290, as described further below. As mentioned above, the secondary therapeutic agent 258 may also be disbursed in the polymer matrix 252 as needed.

In some embodiments, smaller particle sizes of the therapeutic agents 250, 258 may be desirable. A smaller particle size may provide for faster dissolution (dissolve faster in fluid) and form better interconnectivity of the API (active pharmaceutical ingredient) network within the polymer, allowing for improved drug release. In other embodiments, larger particle sizes of the therapeutic agents 250, 258 may be desirable. In an example of an EVA-based system, a large particle size may lead to more sustained release of the therapeutic agents 250, 258 from the polymer matrix 252 in certain embodiments. Larger particle sizes of the therapeutic agents 250, 258 result in larger pores 254, which may allow a greater amount of the therapeutic agents 250, 258 to diffuse from further beneath the surface 256 of the polymer matrix 252 in some embodiments, resulting in more sustained release of the therapeutic agents 250, 258. The average particle size of the therapeutic agents 250, 258 may be about 1 μm to about 100 μm, more specifically about 5 μm to about 60 μm. In one embodiment, the average particle size for meloxicam is around 7.4 μm. A higher surface area to volume ratio of the extended release coating 290 may also result in more sustained release, as elution of the therapeutic agents 250, 258 results in a greater number of pores 254 over the polymer surface 256 to provide easier diffusion of the therapeutic agents 250, 258 from further beneath the surface 256, while also providing a thinner polymer matrix 252 from which the therapeutic agents 250, 258 elute.

For such embodiments, the materials produced are categorized as crystalline solid dispersions, in which the therapeutic agent 250, 258 in the crystalline form is dispersed and physically embedded in the polymer matrix 252. Over an extended time period, the mechanism of the therapeutic agent release involves the dissolution of the therapeutic agent 250, 258 from the polymer matrix 252, leaving behind voids or pores 254 in the polymer matrix 252, while leaving the polymer matrix 252 intact. As further therapeutic agent particles are released from the polymer matrix 252, the pores 254 become interconnected, and the interconnected pores can form channels, which may enhance the release of the therapeutic agent 250, 258 from within the polymer matrix 252. Furthermore, in some embodiments such as at least EVA and PCL systems, the coating surface texture impacts the therapeutic agent release kinetics. For example, a rougher surface texture has more surface area and provides channels near the surface of the coating that allow for fluid uptake, resulting in more API dissolution and release. In another example, a smoother surface texture may provide for improved insertion of the cannula, aesthetics, and coating integrity. Accordingly, the surface texture of the coating may be adjusted in the design of the system to facilitate achieving a desired release rate.

In one exemplary embodiment, a target release profile of the present disclosure provides between approximately 0.75 mg of released meloxicam and 1.75 mg of released meloxicam over 14 days per 9 mm device. The formulation may include a polymer matrix with an EVA grade of 2803A, 2820A, 3325A, or 4030AC, for example. Where the polymer matrix 252 has an EVA grade of 2803A, the formulation may include a vinyl acetate percentage of about 28% with a melt index of 3 dg/min. Where the polymer matrix 252 has an EVA grade of 2820A, the formulation may include a vinyl acetate percentage of around 28% with a melt index of 25 dg/min. Where the polymer matrix 252 has an EVA grade of 3325A, the formulation may include a vinyl acetate percentage of around 33% with a melt index of around 43 dg/min. Where the polymer matrix 252 has an EVA grade of 4030AC, the formulation may include a vinyl acetate percentage of about 40% with a melt index of around 55 dg/min. Table 1 provides exemplary formulations including EVA that substantially achieve this target release profile.

TABLE 1
Formulations of EVA polymer matrices to substantially
achieve a target release profile.

| | Vinyl | Melt | | Meloxicam | Coating |
|---|---|---|---|---|---|
| EVA | Acetate | Index | Polymer | Loading | Thickness |
| Grade | (%) | (dg/min) | Level | (wt. %) | (μm) |
| 2803A | 28 | 3 | Mid | 70 | 51.8 |
| 2803A | 28 | 3 | High | 55 | 94.4 |
| 2803A | 28 | 3 | High | 70 | 66.6 |
| 2820A | 28 | 25 | Mid | 70 | 87.1 |
| 2820A | 28 | 25 | High | 70 | 62.4 |
| 3325A | 33 | 43 | High | 55 | 103.0 |
| 3325A | 33 | 43 | High | 70 | 88.6 |
| 4030AC | 40 | 55 | Mid | 70 | 40.4 |
| 4030AC | 40 | 55 | High | 70 | 82.2 |

The therapeutic coating 290 may be loaded with a desired amount of the therapeutic agents 250, 258, such as about 20-75 wt. %, more specifically about 30-75 wt. %, more specifically 30-65 wt. %, more specifically about 35-60 wt. %, more specifically about 40-55 wt. %, and more specifically about 45-55 wt. %. As shown in Table 1, above, for example, for EVA coatings, the therapeutic coating 290 may include a meloxicam loading of about 55-70 wt. %, which corresponds to about 42-58 vol. % meloxicam, respectively. To mitigate the risk of poor coating integrity so that the polymer matrix 252 remains substantially intact as the meloxicam is released, the preferred loading weight percentage of meloxicam may be about 55 wt. % or less, so that over 50 vol. % of the coating is comprised of EVA, for example. In other embodiments including a PCL-meloxicam coating, the therapeutic coating 290 may include a meloxicam loading of about 30-65 wt. %, more specifically about 50 wt. %.

The coating thickness of the therapeutic coating 290 may be from about 20 μm to about 200 μm, more specifically about 40 μm to about 160 μm. For example, the coating thickness may be about 20 μm, about 40 μm, about 60 μm, about 80 μm, about 100 μm, about 120 μm, about 140 μm, about 160 μm, about 180 μm, or about 200 μm. The coating thickness may vary based on the average particle size of the therapeutic agents 250, 258. In certain embodiments, the coating thickness may be about 3 to about 30 times larger than the average particle size of the therapeutic agents 250, 258, such that the therapeutic coating 290 can accommodate several “layers” of therapeutic agent particles 250, 258 beneath the surface 256.

In some embodiments, the thicknesses of the coatings 290 may depend on the polymer-meloxicam formulation and catheter geometry, including liner thickness and catheter length. For example, the range of an exemplary PCL-meloxicam coating thicknesses may be about 48-224 μm, including about 48-101 μm for a 13 mm catheter, about 67-139 μm for a 9 mm catheter, or about 96-224 μm for a 6 mm catheter. In embodiments limiting the overall diameter of the catheter to 1 mm or less, the maximum coating thicknesses, depending on the liner thickness as discussed further herein, may be about 160-210 μm for liner thicknesses of about 0.07-0.12 mm.

In certain embodiments, and as shown in FIG. 2B, the therapeutic coating 290 may be at least partially covered by an optional barrier layer 292. The barrier layer 292 may be configured to facilitate delay or otherwise inhibit elution of the therapeutic agents 250, 258 from the underlying therapeutic coating 290. The barrier layer 292 may comprise any suitable material, such as a thick layer of silicone, for example. In other embodiments, a thin silicone coating applied to the catheters does not substantially impact the plasma profiles and release of the therapeutic agent.

### Methods

### Solvent-Based

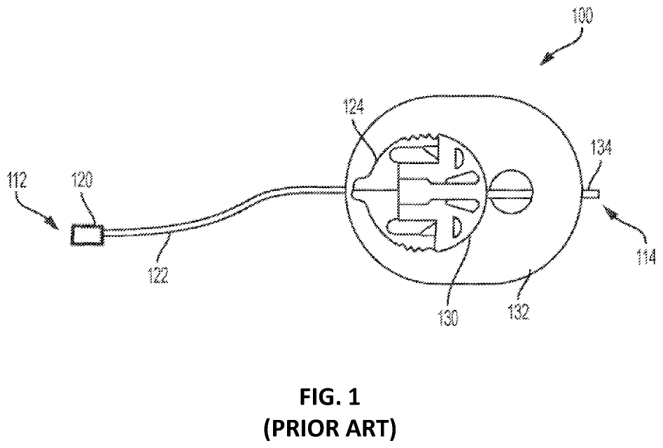

A solvent-based method may be used to produce the therapeutic coating 290 of FIGS. 1-2B. In such a method, a desired polymer and one or more therapeutic agents are dissolved in a solvent. As discussed further herein, the solvent may include chloroform or another solvent suitable for dissolving the desired polymer and therapeutic agents.

The solution containing the solvent, dissolved polymer, and therapeutic agent is then applied to the outer surface 235 of the infusion catheter 234 of FIGS. 1, 2A, and 2B by spraying, painting, dipping, or otherwise covering the infusion catheter 234. In the context of dipping, for example, the dip count, dwell time in the coating solution, dip speed, and dry or dwell time between dips may be varied to achieve the desired coating thickness of the therapeutic coating 290 without excessive displacement of the coating solution. For example, the infusion catheter 234 may be dipped from one time (to produce a relatively thin therapeutic coating 290) to five times or more (to produce a relatively thick therapeutic coating 290). Also, the infusion catheter 234 may be allowed to dry between repeated dips to allow each layer to reach a more durable state before being reintroduced into the coating solution, which could displace or otherwise disturb the previous layer. As another example, the infusion catheter 234 may be dipped at speeds from about 1 in./min. to about 20 inches/min.

Finally, the solvent is permitted to evaporate from the solution, leaving behind the therapeutic coating 290 of the polymer matrix 252 and therapeutic agents 250, 258 on the infusion catheter 234.

### Melt-Based Molding

A melt-based molding method may be used to produce the therapeutic coating 290 of FIGS. 1-2B. In such a method, a desired polymer, such as EVA, is melted. Therapeutic agent particles 250, 258, such as meloxicam particulates, are mixed into the polymer melt. The combined therapeutic agent particles 250, 258 and polymer melt are kept below the melting point of the therapeutic agents 250, 258 to avoid melting and recrystallizing of the therapeutic agents 250, 258. This low temperature mitigates the risk of compromising the cohesive characteristics of the polymer matrix 252 while ensuring the therapeutic agent particles 250, 258 remain at a larger size. The polymer melt with therapeutic agent particles 250, 258 are formed into a shape by compression molding, injection molding, or another molding process as described further herein, and permitted to solidify to form a film. The film may then be applied to the infusion catheter 234 of FIGS. 1-2B via adhesion. In other embodiments, the polymer melt may be applied to and allowed to solidify upon the catheter 234 via overmolding.

### Hot Melt Extrusion Method

In some embodiments, single screw or twin screw hot melt extrusion may be utilized to form the therapeutic coating 290 of FIGS. 1-2B. Extrusion may be performed using an extruder, or a barrel with a single screw or twin screws which rotate to transport and mix a desired melted polymer and a therapeutic agent 250, 258 through the barrel and out of an orifice which shapes the mixed polymer and therapeutic agent 250, 258 into a desired shape, such as a rod. Frictional heating or externally sourced heat may be used for reaching and/or maintaining the melt status of the polymer. In twin screw extrusion, high-speed energy input twin-screw extruders or low-speed late fusion twin-screw extruders may be utilized. Twin screw extruders may be co-rotating or counter-rotating.

In one embodiment, a two-stage process is utilized. The twin screw extrusion is used for compounding to produce pellets of homogenous polymer-therapeutic agent material. In a second extrusion stage, the pellets of polymer-therapeutic agent material are fed into a single-screw extrusion process as the shape forming step, such as over-extrusion for applying a polymer-therapeutic coating onto tubing. In other embodiments, the secondary stage could be micro-injection molding, in which the pellets of polymer-therapeutic agent are fed into a hopper and injection molded to form a catheter or catheter coating 234 via overmolding or two-shot molding.

For production of bi-layer drug eluting catheters, twin-screw extrusion may be used for compounding to mix a polymer and a therapeutic agent to produce homogenous polymer-therapeutic pellets at the target therapeutic agent loading level (wt. %). A single screw extrusion process may be used as a shape forming step. The options for the single screw extrusion may include (1) over-extrusion, where the polymer-therapeutic is applied as a coating onto tubing (polymer liner and tubing produced separately); and (2) co-extrusion of bi-layer tubing where the inner layer (liner) is a polymer without a therapeutic agent (e.g., HDPE, Tritan, PTFE, TPU, FEP, etc.) and the outer layer, or coating, is a polymer-therapeutic coating. The coated tubing is an intermediate product and requires post-processing for forming the catheter. The post-processing includes: (1) cutting the coated tubing to the target length for a 6 mm, 9 mm, or 13 mm catheter; (2) completing a flaring process at the proximal end and press fitting the bushing to the proximal end; and (3) completing a catheter tipping process to form the taper of the outer diameter and the inner diameter of the catheter at the distal end. The bi-layer catheter would then be assembled as part of an infusion set.

Injection Molding Method for Tubes and other Shapes

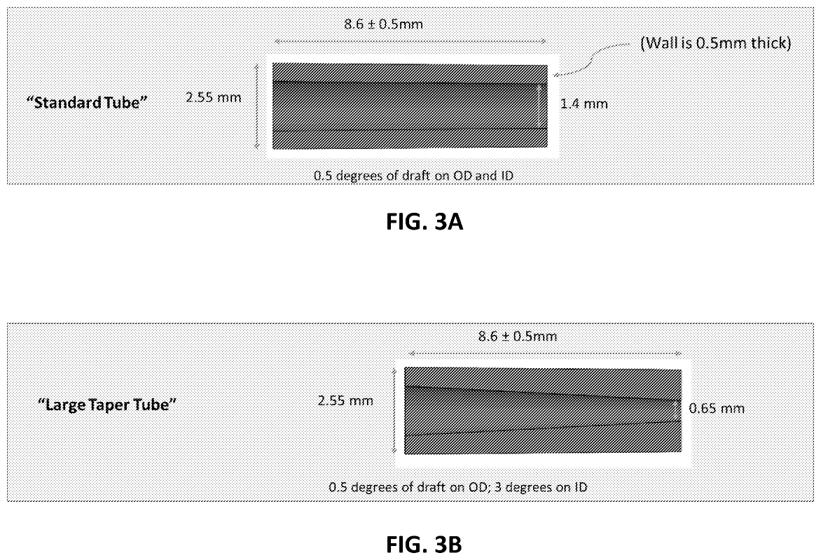

In some embodiments, an injection molding method may be utilized to form molded shapes of polymer-API material. Two exemplary designs of tubes are illustrated in FIGS. 3A and 3B, including a standard tube as shown in FIG. 3A or a tapered tube as shown in FIG. 3B. The exemplary standard tube has an outer diameter measuring about 2.55 mm with 0.5 degrees of draft, an inner diameter measuring about 1.4 mm with 0.5 degrees of draft, and a length measuring about 8.6 mm with a 0.5 mm differential. The wall of the tube defined by the inner diameter and the outer diameter may be about 0.5 mm thick. The exemplary tapered tube has an outer diameter measuring about 2.55 mm with 0.5 degrees of draft, an inner diameter having a tip measuring about 0.65 mm with 3 degrees of draft, and a length measuring about 8.6 mm with 0.5 degrees of draft. Additional examples of injection molded controlled-release systems may be found in International PCT Application No. PCT/US2020/028195 to CARTER, et al., published Oct. 22, 2020, and titled “INFUSION HEAD WITH CONTROLLED RELEASE OF SECONDARY DRUG”, the disclosure of which is hereby expressly incorporated by reference in its entirety.

### Micro-Injection Molding Method for Coated Catheters

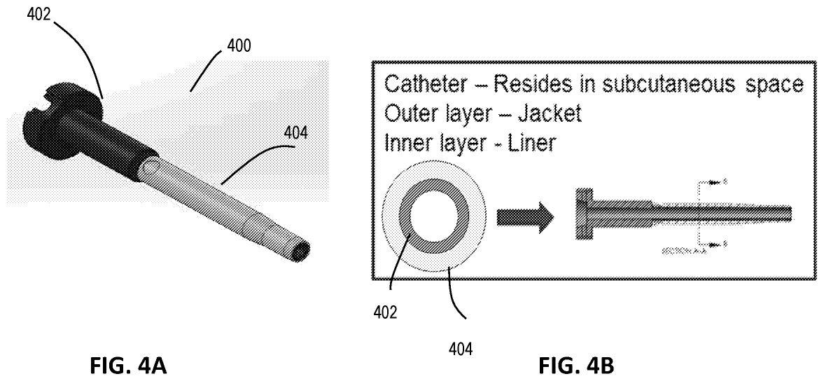

A micro-injection molding method may involve a two-shot mold or over-mold. The mold may be designed to produce a catheter that includes the bushing at the proximal end and the taper at the distal end, and therefore, not require post-processing steps. The first shot may be a polymer without a therapeutic agent as the material for the liner and the bushing (e.g., HDPE, etc.), and the second shot may be a polymer loaded with the therapeutic agent. Specifically, the polymer-meloxicam pellets produced from a twin-screw extrusion compounding step may be fed into the hopper of a micro-injection molding machine for the second shot. The final product from the two-shot micro-injection molding process may be a bi-layer drug-eluting catheter, which may be assembled as part of an infusion set. An exemplary catheter 400 produced by micro-injection molding is illustrated in FIGS. 4A and 4B. Catheter 400 includes a first shot 402 including a liner and bushing and a second shot 404 which is a coating composed of polymer and therapeutic agent.

### EXAMPLES

The following examples describe the manner, process of making, and/or process of using the present disclosure and are intended to be illustrative rather than limiting.

### Solvent-Based Example 1

Quantities of EVA and meloxicam were dissolved in a chloroform solvent. Using a pipette, the EVA-meloxicam solution was dispersed onto a Teflon surface, and the chloroform was then allowed to evaporate, forming a film or coupon. Once the chloroform was evaporated, the film was peeled from the surface of the Teflon while remaining substantially intact. The films were each immersed in phosphate-buffered saline (PBS) to yield a target therapeutic agent concentration, assuming 100% therapeutic agent release rate. The volume of PBS used varied depending on the measured film weight. The coupons included a therapeutic agent loading of 40 wt. %, 55 wt. %, or 70 wt. %. Each coupon was subcutaneously implanted in a rat. The coupons may or may not be siliconized immediately prior to implantation. For a two-week testing period, whole blood was drawn from the rats, plasma was separated from the whole blood, and plasma meloxicam levels were measured by HPLC-MS.

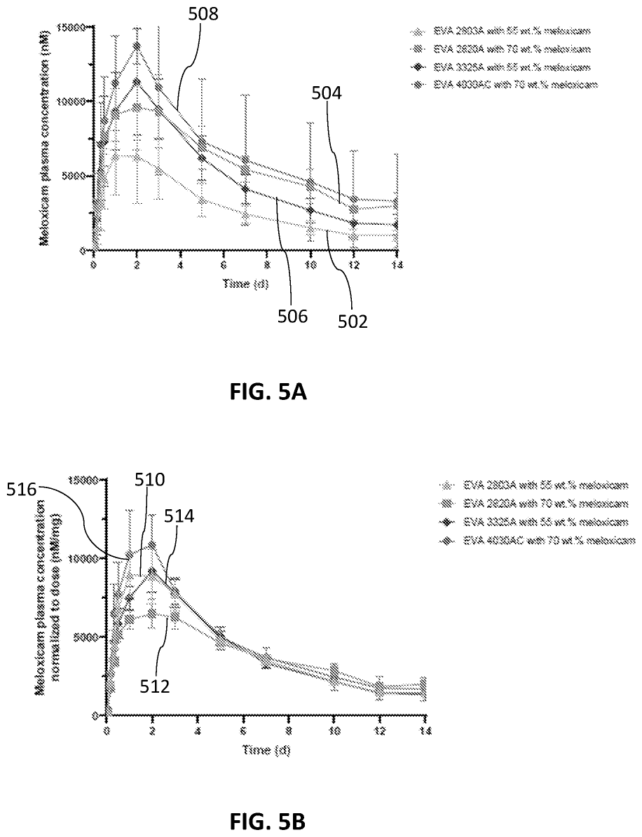

FIG. 5A provides the average release profiles of the coupon formulations in vivo following subcutaneous implantation over 14 days. Specifically, FIG. 5A provides the average level of meloxicam plasma concentration in the tested rats over a period of 14 days, wherein each average corresponds to a specified formulation. For example, line 502 interconnecting the triangle-shaped data points illustrates the average level of meloxicam plasma concentration from a rat treated with a film having an EVA grade of 2803A comprising a meloxicam loading of 55 wt. %. Line 504 interconnecting the square-shaped data points illustrates the average level of meloxicam plasma concentration from a rat treated with a film having an EVA grade of 2820A with a meloxicam loading of 70 wt. %. Line 506 interconnecting the diamond-shaped data points illustrates the average level of meloxicam plasma concentration from a rat treated with a film having an EVA grade of 3325A with a meloxicam loading of 55 wt. %. Line 508 interconnecting the circle-shaped data points illustrates the average level of meloxicam plasma concentration from a rat treated with a film having an EVA grade of 4030AC with a meloxicam loading of 70 wt. %.

The dosing of meloxicam in each of the four formulations may differ according to the meloxicam loading level and thickness of the coating. FIG. 5B provides the average normalized-to-dose level of meloxicam plasma concentration in the tested rats over a period of 14 days. For example, line 510 interconnecting the triangle-shaped data points illustrates the average normalized-to-dose meloxicam plasma concentration from a rat treated with a film having an EVA grade of 2803A with a meloxicam loading of 55 wt. %. Line 512 interconnecting the square-shaped data points illustrates the average normalized-to-dose meloxicam plasma concentration from a rat treated with a film having an EVA grade of 2820A with a meloxicam loading of 70 wt. %. Line 514 interconnecting the diamond-shaped data points illustrates the average normalized-to-dose meloxicam plasma concentration from a rat treated with a film having an EVA grade of 3325A with a meloxicam loading of 55 wt. %. Line 516 interconnecting the circle-shaped data points illustrates the average normalized-to-dose meloxicam plasma concentration from a rat treated with a film having an EVA grade of 4030AC with a meloxicam loading of 70 wt. %.

As shown in FIG. 5B, the release profile of each film may vary relative to other films over the first three days dependent on the EVA formulation used. However, the release profiles for the remaining days are substantially the same. As such, for treatment purposes, the formulation may be altered to meet individualized goals. For example, as shown by line 516, the film having an EVA grade of 4030AC with a meloxicam loading of 70 wt. % had the highest peak meloxicam concentration. Additionally, as shown by line 512, the film having an EVA grade of 2820A with a meloxicam loading of 70 wt. % had the lowest peak meloxicam concentration. Where a high initial burst of meloxicam elution is desired, a formulation of EVA 4030AC with a meloxicam loading of 70 wt. % consistent with line 516 may be used. Where a low initial burst of meloxicam elution is desired, a formulation of EVA 2820A with a meloxicam loading of 70 wt. % consistent with line 512 may be used.

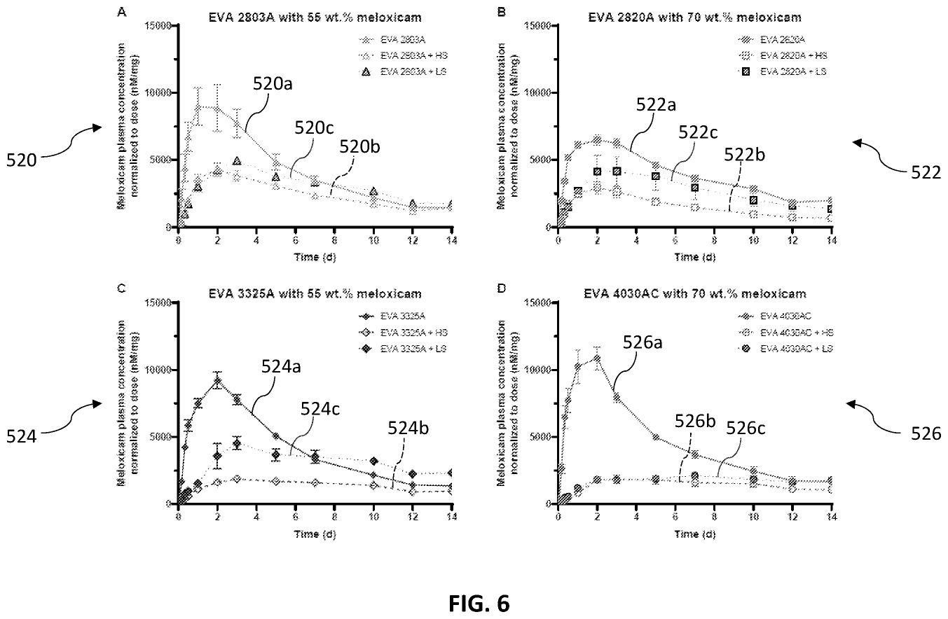

As mentioned above, the film may be coated with silicone before being implanted. FIG. 6 illustrates exemplary effects of such siliconization by dipping the films in silicone oil to form thick silicone layers, as detailed below. For example, graph 520 illustrates the effects of silicone oil on the normalized-to-dose release profile of films having an EVA grade of 2803A with a meloxicam loading of 55 wt. %, wherein the solid line 520a illustrates the average release profile of films without siliconization, the dashed line 520b illustrates the average release profile of films siliconized with high viscosity oil, and the dotted line 520c illustrates the average release profile of films siliconized with low viscosity oil. Graph 522 illustrates the effects of silicone oil on the normalized-to-dose release profile of films having an EVA grade of 2820 with a meloxicam loading of 70 wt. %, wherein the solid line 522a illustrates the average release profile of films without siliconization, the dashed line 522b illustrates the average release profile of films siliconized with high viscosity oil, and the dotted line 522c illustrates the average release profile of films siliconized with low viscosity oil.

Graph 524 illustrates the effects of silicone oil on the normalized-to-dose release profile of films having an EVA grade of 3325A with a meloxicam loading of 55 wt. %, wherein the solid line 524a illustrates the average release profile of films without siliconization, the dashed line 524b illustrates the average release profile of films siliconized with high viscosity oil, and the dotted line 520c illustrates the average release profile of films siliconized with low viscosity oil. Graph 526 illustrates the effects of silicone oil on the normalized-to-dose release profile of films having an EVA grade of 4030AC with a meloxicam loading of 70 wt. %, wherein the solid line 526a illustrates the average release profile of films without siliconization, the dashed line 526b illustrates the average release profile of films siliconized with high viscosity oil, and the dotted line 526c illustrates the average release profile of films siliconized with low viscosity oil.

The silicone layers (especially the high-viscosity silicone layers of lines 520b, 522b, 524b, and 526b) serve as a diffusion barrier, which results in reduced and/or delayed meloxicam uptake as shown, especially at the initial burst elution as shown in FIG. 6. Thin layers of silicone oil may otherwise be utilized to provide a lesser or negligible barrier effect, while thicker layers of silicone oil may be utilized to control elution of meloxicam as shown.

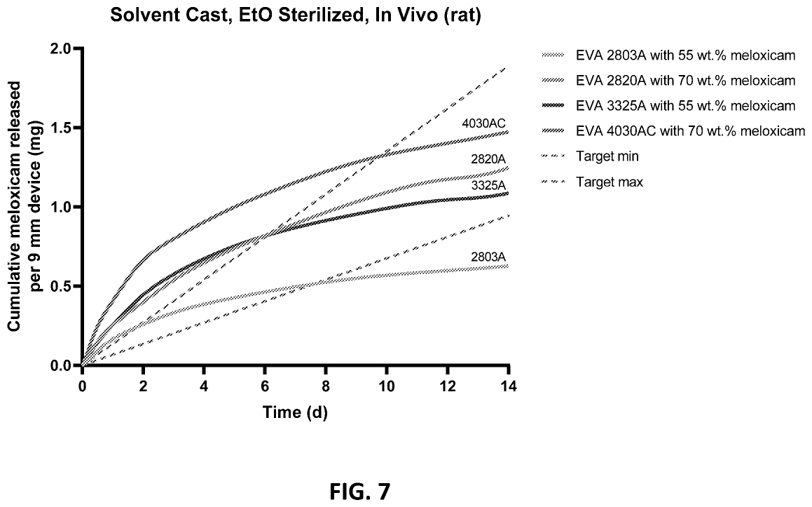

The in vivo release profiles were calculated using a deconvolution model for translating the rat plasma profile concentrations to in vivo release profiles. The deconvolution method is a technique to estimate an input function, which are the release profiles from EVA-meloxicam coupons, given the corresponding input-response function, which are the plasma concentration data from EVA-meloxicam coupons, and the impulse response function, which is the plasma concentration profile following an IV bolus dose, for the system. The key assumptions for this model are as follows: (i) subcutaneous dose showed very fast adsorption and is assumed to behave like an IV dose, and therefore has been used as the impulse response function, (ii) linearity: f(D1+D2)=f(D1)+f(D2), and (iii) time invariance: f(D) has the same shape no matter when D is given. The in vivo release profiles illustrated in FIG. 7 were calculated from the rat plasma concentrations shown in FIG. 5B using a deconvolution model which takes into account absorption and systemic clearance.

### Solvent-Based Example 2

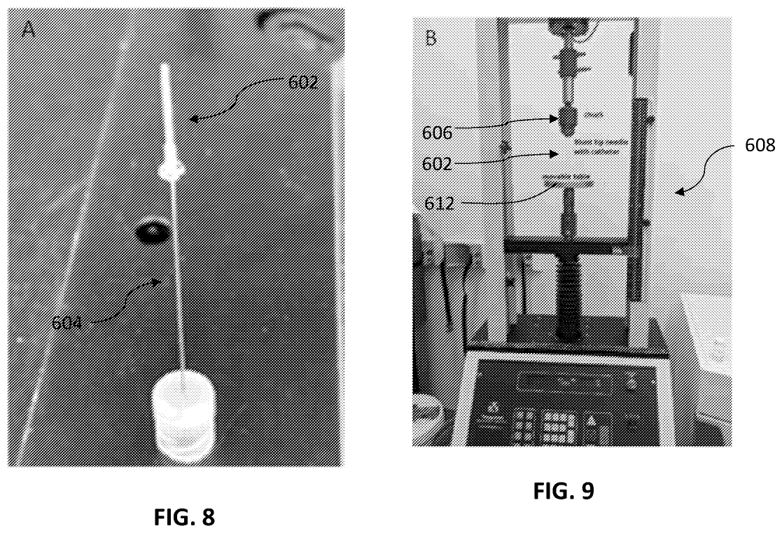

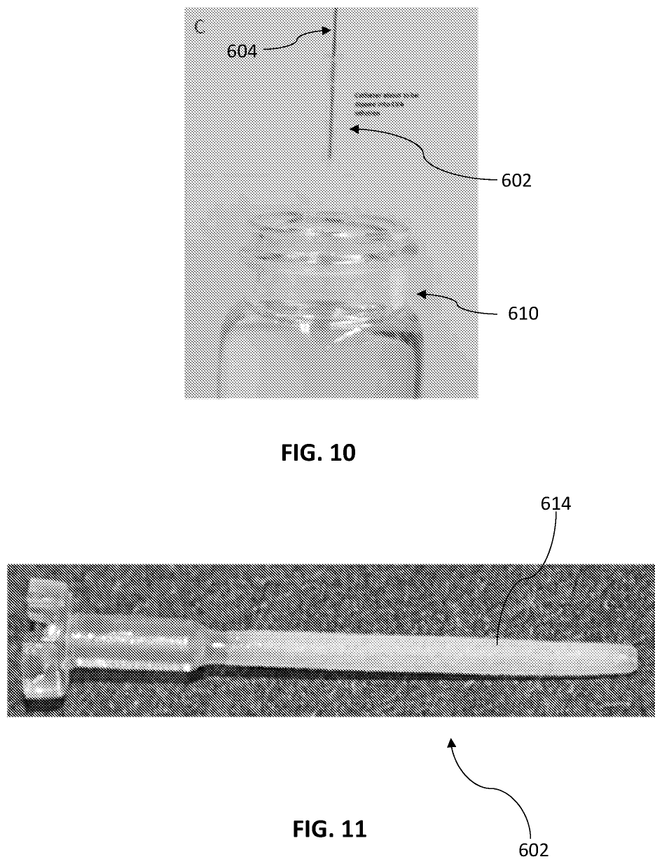

Referring to the example of FIGS. 8-11, to apply the EVA-meloxicam solution to a catheter 602, a dip coating method was utilized. As discussed above, EVA polymer was dissolved in chloroform to reach a target EVA concentration using a calibrated balance and pipettes. Specifically, EVA2820A was prepared at a target concentration of 60 mg/mL, while EVA3325A was prepared at a target concentration of 100 mg/mL. Meloxicam was added to each solution to reach a target concentration. Specifically, meloxicam was added at 70 wt. % to the EVA2820A solution and 55 wt. % to the EVA3325A solution. The solutions were combined in respective vials.

Now referring to FIGS. 8-10, the catheter 602 was mounted to a needle 604 so that the tip of the catheter 602 and the tip of the needle 604 were aligned. The proximal end of the needle 604 was coupled within a chuck 606 of a compression testing assembly 608, specifically a Thwing Albert test frame. A vial 610 containing the desired solution was disposed on a moveable table 612 of the compression tester 608. The compression tester 608 actuated the moveable table 612 so that the vial 610 was lifted until the catheter 602 was submerged within the solution. The catheter 602 was dipped at a rate of 18 in./min. FIG. 11 illustrates an exemplary catheter 602 having an EVA-meloxicam coating 614 resulting from the dip method as discussed herein.

### Melt-Based Molding Example 3

In this example, sheets of meloxicam and ethyl vinyl acetate (EVA) were formed by melt compounding and compression molding with nominal thicknesses of 90 μm, 120 μm, or 150 μm. Mini tensile bars were die cut from these sheets using a clicker press, then the actual thickness of each mini tensile bar was measured using a micrometer.

The mini tensile bars were pulled in triplicate in an Instron with a 5 in/min extension rate at 23° C. and 50% RH to measure the stress at maximum load, the strain at maximum load, and the Young's Modulus of each mini tensile bar.

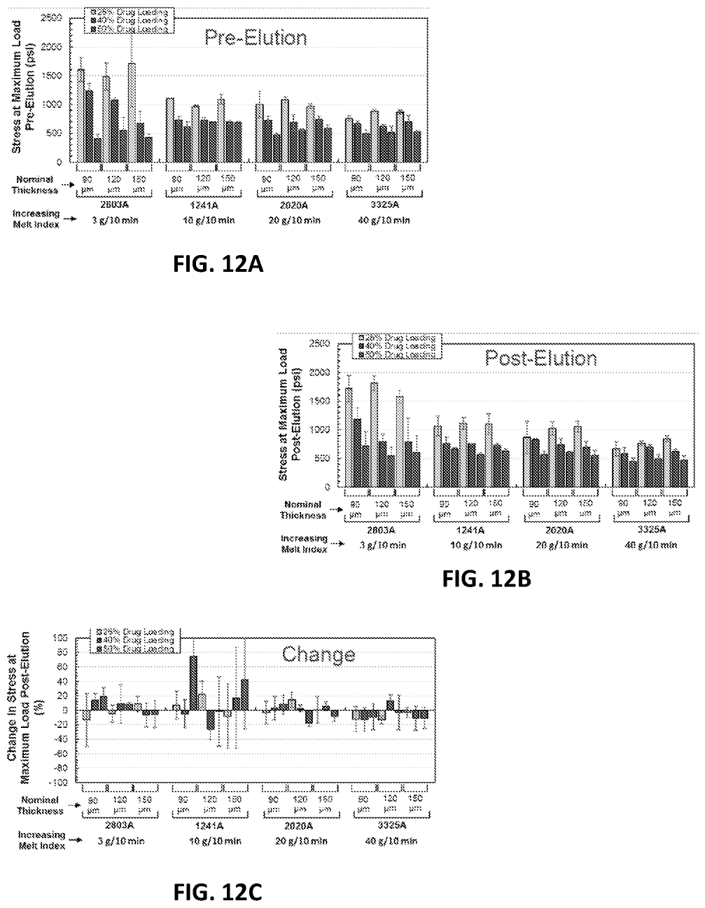

The stress measurements are set forth in FIGS. 12A, 12B, and 12C. FIG. 12A provides the average stress at maximum load for each category of mini tensile bars having the same characteristic nominal thickness, polymer type and corresponding melt index, and therapeutic agent loading pre-elution. FIG. 12B provides the average stress at maximum load for each category of mini tensile bars having the same characteristic nominal thickness, polymer type and corresponding melt index, and therapeutic agent loading post-elution. FIG. 12C provides the average change in stress at maximum load between pre-elution and post-elution for each category of mini tensile bars having the same characteristic nominal thickness, polymer type and corresponding melt index, and therapeutic agent loading.

The following effects are observed from the example of FIGS. 12A, 12B, and 12C. Increasing the therapeutic agent load decreased the tensile strength for all other characteristic types, as increase of therapeutic agent particles may disrupt the polymer chain interdigitation of the surrounding polymer matrix. Additionally, tensile strength tended to decrease as the melt index increased, with mini tensile bars comprised of Ateva® 2803A showing the highest tensile strength and mini tensile bars comprised of Ateva® 3325A showing the lowest tensile strength. However, these melt index effects were only observable in mini tensile bars having the lowest therapeutic agent loading at 25%; the addition of meloxicam weakened the polymer enough that the differences between polymer types were no longer observable. For the majority of mini tensile bar characteristic types, there were few observable trends in the effect of therapeutic agent elution on the average tensile strength of the mini tensile bar. Typically, a change of 20% or less was observed.

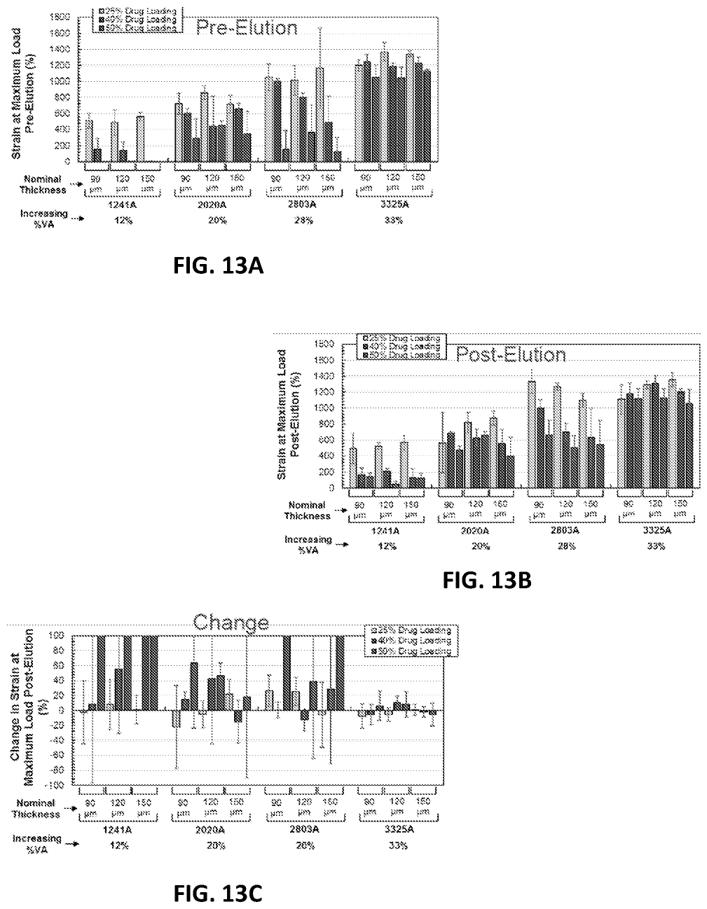

The strain measurements are set forth in FIGS. 13A, 13B, and 13C. FIG. 13A provides the average strain at maximum load for each category of mini tensile bars having the same characteristic nominal thickness, polymer type and corresponding percentage of vinyl acetate, and therapeutic agent loading pre-elution. FIG. 13B provides the average strain at maximum load for each category of mini tensile bars having the same characteristic nominal thickness, polymer type and corresponding percentage of vinyl acetate, and therapeutic agent loading post-elution. FIG. 13C provides the average change in strain at maximum load between pre-elution and post-elution for each category of mini tensile bars having the same characteristic nominal thickness, polymer type and corresponding percentage of vinyl acetate, and therapeutic agent loading.

The following effects are observed from FIGS. 13A, 13B, and 13C. Increasing the therapeutic agent load decreased the strain at maximum load for all other characteristic types, as increase of therapeutic agent particles may disrupt the polymer chain interdigitation of the surrounding polymer matrix. As the percentage of vinyl acetate increased, the strain at maximum load tended to increase. For example, as shown, the mini tensile bars comprised of Ateva® 1241A averaged the lowest strain at maximum load, while the mini tensile bars comprised of Ateva® 3325A averaged the highest strain at maximum load. This trend between strain and percentage of vinyl acetate was observable across therapeutic agent loadings. For mini tensile bars within that displayed extremely low strain at maximum load pre-elution (i.e., Ateva® 1241A with 50% therapeutic agent loading; Ateva® 12471A with a nominal thickness of 150 μm and 40% therapeutic agent loading; Ateva® 2803A with a nominal thickness of 90 μm or 150 μm and 50% therapeutic agent loading), an increase in strain was observed post-elution. Because the pre-elution strain was so low, dramatic changes in strain between pre-elution and post-elution are observed in FIG. 13C.

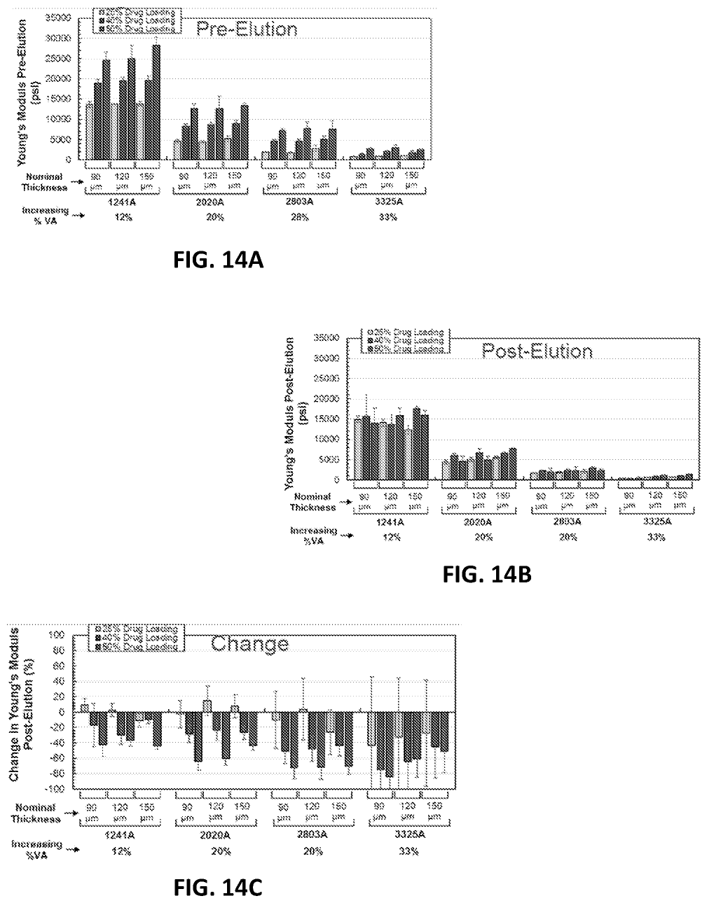

The Young's Modulus measurements are set forth in FIGS. 14A, 14B, and 14C. FIG. 14A provides the average Young's Modulus for each category of mini tensile bars having the same characteristic nominal thickness, polymer type and corresponding percentage of vinyl acetate, and therapeutic agent loading pre-elution. FIG. 14B provides the average Young's Modulus for each category of mini tensile bars having the same characteristic nominal thickness, polymer type and corresponding percentage of vinyl acetate, and therapeutic agent loading post-elution. FIG. 14C provides the average change in Young's Modulus between pre-elution and post-elution for each category of mini tensile bars having the same characteristic nominal thickness, polymer type and corresponding percentage of vinyl acetate, and therapeutic agent loading.

The following effects are observed from FIGS. 14A, 14B, and 14C. Increasing the therapeutic agent load increased the Young's Modulus for all samples, as increase of therapeutic agent particles may disrupt the polymer chain interdigitation of the surrounding polymer matrix. As the percentage of vinyl acetate increased, the Young's Modulus tended to decrease. For example, as shown, the mini tensile bars comprised of Ateva® 1241A averaged the highest Young's Modulus value, while the mini tensile bars comprised of Ateva® 3325A averaged the lowest Young's Modulus value. As shown in FIG. 14C, exposure to the elution buffer generally cased the Young's Modulus to decrease as the mini tensile bars became less brittle. This effect was greater for samples with high therapeutic agent loading and high percentage of vinyl acetate. As the therapeutic agent elutes from the polymer, the polymer chains become hydrated, especially where the polymer is more hydrophilic with a high percentage of vinyl acetate, such as Ateva® 3325A.

### Melt-Based Molding Example 4

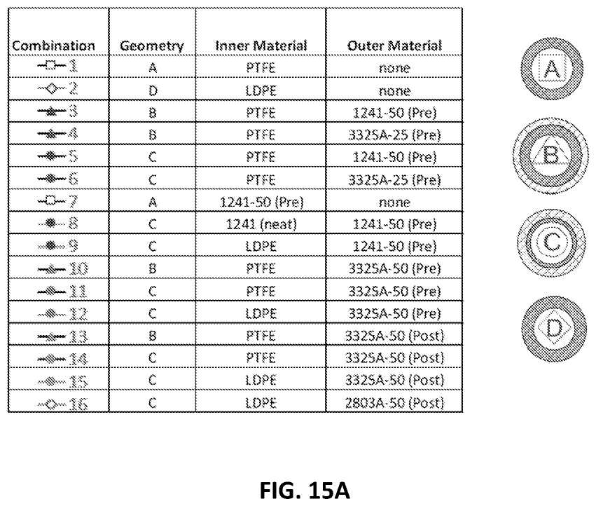

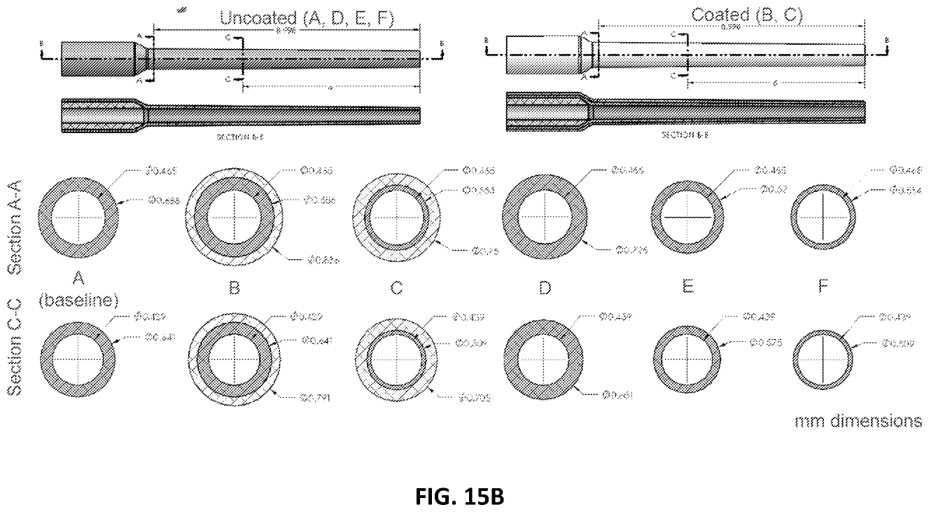

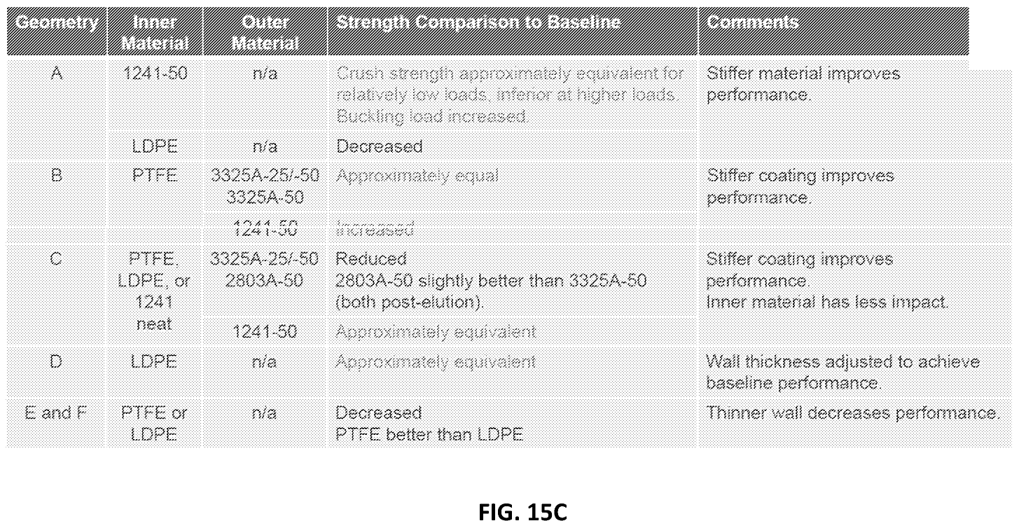

A finite element analysis was performed to model various catheter formulations and geometries to compare responses to mechanical loading, including responses to radial crush and axial buckling. Sixteen different combinations of catheter geometries and materials were formed according to the provided information of FIG. 15A. Additionally, catheters were formed according to geometries “E” and “F” as shown in FIG. 15B, wherein each of these catheters comprised an inner material of at least one of polytetrafluoroethylene (PTFE) and low-density polyethylene (LDPE). Geometries “A”; “B”; “C”; “D”; “E”; and “F” were formed according to the specifications given in FIG. 15B, wherein dimensions are shown in millimeters, geometries “B” and “C” included outer material, while geometries “A”; “D”; “E”; and “F” did not include outer material. The inner material comprised at least one of PTFE; LDPE; Ateva® 1241A with no therapeutic agent loading (“1241 (neat)”); or Ateva® 1241A with 50% therapeutic agent loading (“1241-50”) pre-elution as shown and described in FIG. 15A. For catheters including an outer material, the outer material comprised at least one of Ateva® 1241A with 50% therapeutic agent loading pre-elution; Ateva® 3325A with 25% therapeutic agent loading (“3325A-25”) pre-elution; Ateva® 3325A with 50% therapeutic agent loading (“3325A-50”) pre-elution; Ateva® 3325A with 50% therapeutic agent loading post-elution, and Ateva® 2803A with 50% therapeutic agent loading (“2803A-50”) post-elution.

To analyze radial crush responses for each tested catheter, a rigid, stainless steel 1 mm diameter cylinder was displaced about 0.2 mm into a side wall of the catheter. The load was applied 6 mm from the tip of the tested catheter. To analyze axial buckling for each tested catheter, the entire bottom surface of the tested catheter was fixedly coupled to a fixed surface, and then an axial load was applied on the top surface of the tested catheter. The results of such testing can be found in FIG. 15C, wherein it was found for this experiment that stiffer inner material improves performance compared to less stiff inner material and that stiffer coating material improves performance compared to less stiff inner material. Notably, the stiffness of the coating lessens the impact of the inner material in this example. Additionally, thinner inner material walls decreased performance during the crushing and buckling testing in this example. Ultimately, the findings concluded that a catheter including a coating may be added to an existing PTFE catheter or thickened LDPE catheter and perform as well as conventional PTFE catheters in crush and/or buckling incidents.

### Melt-Based Molding Example 5

In this example, film coupons were prepared using EVA 3325A with a therapeutic agent loading of 55 wt. % meloxicam. Each coupon was prepared at a measured thickness of 90 μm with a meloxicam particle size of either d90=26 μm or d90=50 μm. For elution testing, each coated monofilament sample was immersed in phosphate buffered saline having a pH of 7.4 at sink conditions, incubated at 37° C. and shaking at 50 rpm for the duration of a 14-day study. At predetermined points throughout the elution study, aliquots were sampled from the solution with replacement, and meloxicam concentration was measured in the aliquots by absorbance.

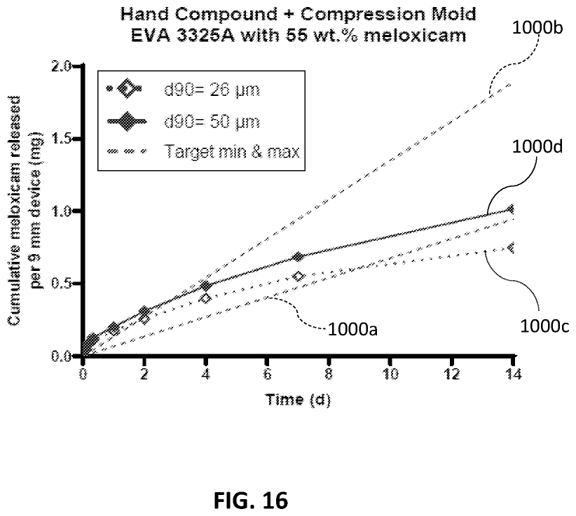

FIG. 16 illustrates the average therapeutic agent release profile of the prepared coupons according to the measured meloxicam particle size. The dashed line 1000a illustrates the minimum target therapeutic agent release profile, while the dashed line 1000b illustrates the maximum target therapeutic agent release profile. Line 1000c interconnecting the hollow diamond-shaped data points illustrates the average therapeutic agent release profile for coupons having a meloxicam particle size of d90=26 μm, and line 1000d interconnecting the shaded diamond-shaped data points illustrates the average therapeutic agent release profile for coupons having a meloxicam particle size of d90=50 μm. As shown, the coupons having a meloxicam particle size of d90=50 μm most closely fits the target therapeutic agent release profile.

### Over-Extrusion Coated Monofilament Example 6

In this example, EVA-meloxicam-coated nylon monofilaments were manufactured using a two-step hot melt extrusion process including a twin-screw extrusion compounding step wherein EVA 3325A and meloxicam were mixed to produce homogenous EVA-meloxicam pellets having a 55 wt. % meloxicam loading. The two-step hot process further included a single-screw extrusion shape-forming step. During the shape-forming step, the EVA-meloxicam pellets were fed into a single-screw extruder having a cross-head, which was used to apply a 100 μm thick coating of EVA-meloxicam onto a nylon monofilament having a 0.73 mm diameter, resulting in an EVA-meloxicam-coated nylon monofilament having a final overall diameter of 0.93 mm.

Coated monofilament samples were tested after both the twin screw extrusion step and the single screw extrusion step. All of the coated monofilament samples were unsterilized. For elution testing, each coated monofilament sample was immersed in phosphate buffered saline having a pH of 7.4 at sink conditions, incubated at 37° C. and shaking at 50 rpm for the duration of a 14-day study. At predetermined points throughout the elution study, aliquots were sampled from the solution with replacement. The concentration of meloxicam was measured in each aliquot by a meloxicam potency assay.

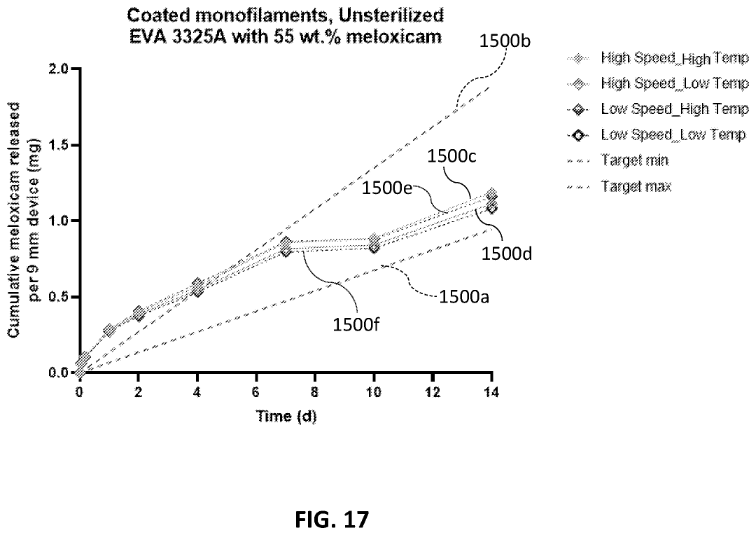

FIG. 17 illustrates the average therapeutic agent release profile of the prepared coated monofilament. The cumulative release profiles illustrated were normalized to the surface area of a 9 mm catheter. The dashed line 1500a illustrates the minimum target therapeutic agent release profile, while the dashed line 1500b illustrates the maximum target therapeutic agent release profile. Line 1500c illustrates the average therapeutic agent release profile of monofilament samples prepared with high speed and high temperature extrusion. Line 1500d illustrates the average therapeutic agent release profile of monofilament samples prepared with high speed and low temperature extrusion. Line 1500e illustrates the average therapeutic agent release profile of monofilament samples prepared with low speed and high temperature extrusion. Line 1500f illustrates the average therapeutic agent release profile of monofilament samples prepared with low speed and low temperature extrusion. As illustrated, every average monofilament sample release profile met the target therapeutic agent target release over 14 days.

### Over-Extrusion Coated Monofilament Example 7

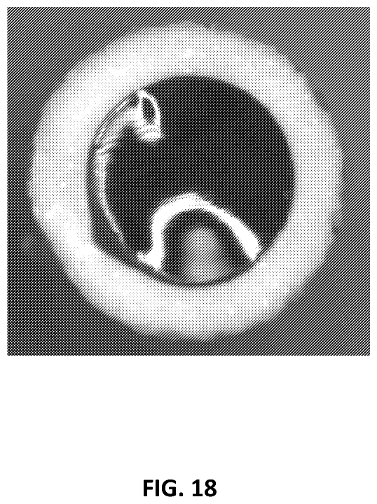

In this example, nylon monofilaments coated with polycaprolactone (“PCL”) and meloxicam coating were manufactured using a two-step hot melt extrusion process. The compounding step was completed using twin-screw extrusion to mix the PCL and meloxicam to produce homogenous PCL-meloxicam pellets. Specifically, the formulation was composed of Purasorb® PC17 (Corbion) with 50 wt. % meloxicam loading. The pellets were then fed into a single-screw extruder for the shape-forming process. The single-screw extruder had a cross-head to apply a PCL-meloxicam coating having a thickness of about 100 μm onto a nylon monofilament having a 0.73 mm diameter, resulting in PCL-meloxicam coated nylon monofilaments having an overall diameter of 0.93 mm. To evaluate the effect of the processing conditions on the meloxicam release from PCL-meloxicam coatings, the high and low values for the screw speed and processing temperature for the single-screw extrusion step were tested as outlined in Table 2 below. The final products were PCL-meloxicam coated nylon monofilaments, and a upon inspection of cross-sections, the coatings were found to be highly concentric as illustrated in FIG. 18.

TABLE 2
Single-screw extrusion processing conditions

| | Target Coating | Screw | Processing |
|---|---|---|---|
| | Thickness | Speed | Temperature |
| Formulation | (μm) | (RPM) | (° C.) |
| PC17 with 50 wt. % | 100-130 | 2 (High) | 145 (High) |
| PC17 with 50 wt. % | 100-130 | 2 (High) | 135 (Low) |
| PC17 with 50 wt. % | 100-130 | 1 (Low) | 145 (High) |
| PC17 with 50 wt. % | 100-130 | 1 (Low) | 135 (Low) |

From a solvent extraction and meloxicam potency assay, the measured drug loading for all of the samples was approximately 49 wt. % meloxicam, which was consistent with the target nominal loading of 50 wt. % meloxicam. The results indicate that the processing conditions in this example, including screw speed and processing temperature, have minimal to no impact on the meloxicam loading level.

For elution testing, each coated monofilament sample was immersed in phosphate buffered saline having a pH of 7.4 at sink conditions, incubated at 37° C. and shaking at 50 rpm for the duration of a 14-day study. At predetermined points throughout the elution study, aliquots were sampled from the solution with replacement. The concentration of meloxicam was measured in each aliquot by a meloxicam potency assay.

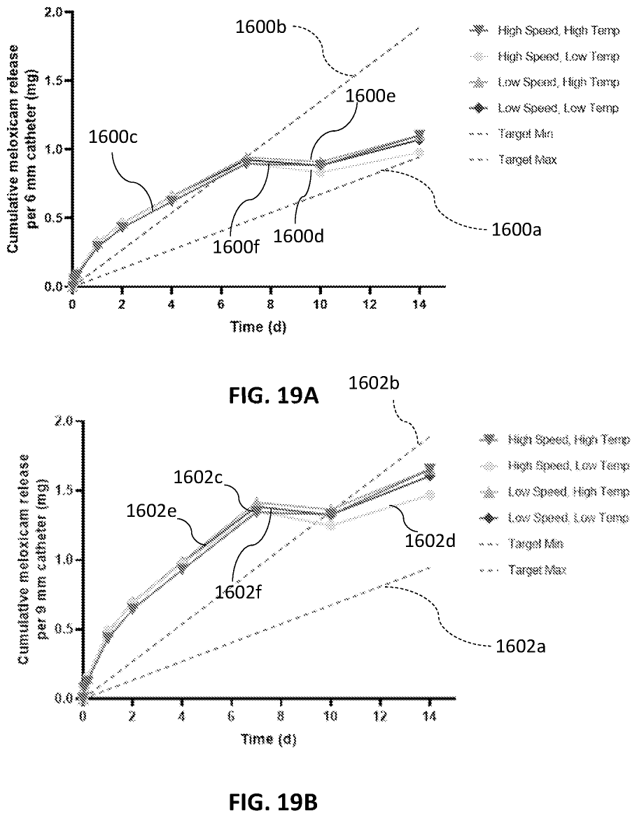

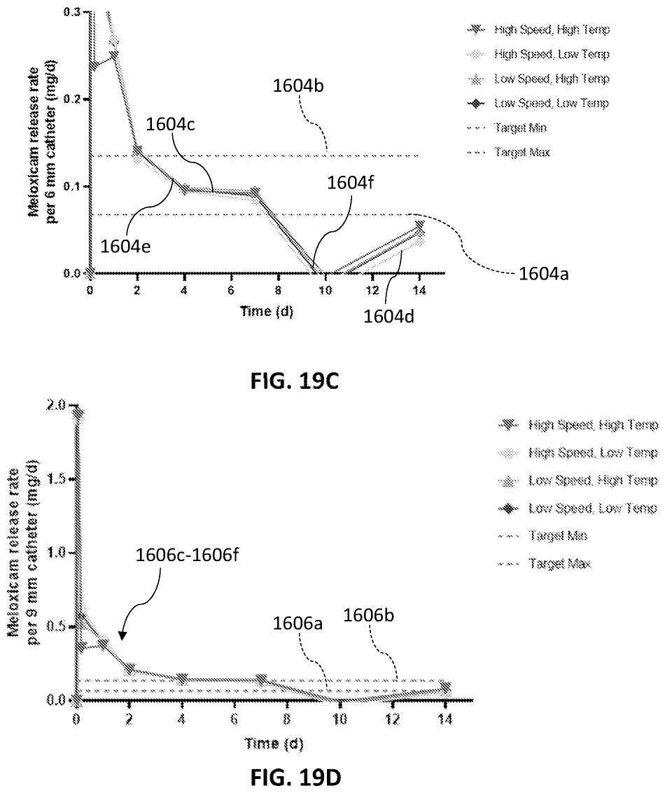

Now referring to FIGS. 19A-D, in vitro release data is presented per 6 mm catheter and 9 mm catheter. The cumulative release of meloxicam per sample was scaled accordingly to the surface areas of a standard commercial catheter with a 100-130 μm coating thickness, such that the surface areas of a 6 mm catheter and a 9 mm catheter are 16.6 mm² and 24.9 mm², respectively. Additionally, the release rates (mg/d) are the derivative of the cumulative release profiles.

Referring specifically to FIG. 19A, illustrative for the cumulative release profile rate per 6 mm catheter, line 1600a illustrates the minimum target therapeutic agent release profile. Line 1600b illustrates the maximum target therapeutic agent release profile. Line 1600c, interconnecting the inverted triangle-shaped data points, illustrates the release profile of samples subjected to high speed and high temperature processes. Line 1600d, interconnecting the circle-shaped data points, illustrates the release profile of samples subjected to high speed and low temperature processes. Line 1600e, interconnecting the upright triangle-shaped data points, illustrates the release profile of samples subjected to low speed and high temperature processes. Line 1600f, interconnecting the diamond-shaped data points, illustrates the release profile of samples subjected to low speed and low temperature processes.

Now referring to FIG. 19B, illustrative for the cumulative release profile per 9 mm catheter, line 1602a illustrates the minimum target therapeutic agent release profile. Line 1602b illustrates the maximum target therapeutic agent release profile. Line 1602c, interconnecting the inverted triangle-shaped data points, illustrates the release profile of samples subjected to high speed and high temperature processes. Line 1602d, interconnecting the circle-shaped data points, illustrates the release profile of samples subjected to high speed and low temperature processes. Line 1602e, interconnecting the upright triangle-shaped data points, illustrates the release profile of samples subjected to low speed and high temperature processes. Line 1602f, interconnecting the diamond-shaped data points, illustrates the release profile of samples subjected to low speed and low temperature processes.

Referring now to FIG. 19C, illustrative for the release rate per 6 mm catheter, line 1604a illustrates the minimum target therapeutic agent release profile. Line 1604b illustrates the maximum target therapeutic agent release profile. Line 1604c, interconnecting the inverted, triangle-shaped data points, illustrates the release profile of samples subjected to high speed and high temperature processes. Line 1604d, interconnecting the circle-shaped data points, illustrates the release profile of samples subjected to high speed and low temperature processes. Line 1604e, interconnecting the upright triangle-shaped data points, illustrates the release profile of samples subjected to low speed and high temperature processes. Line 1604f, interconnecting the diamond-shaped data points, illustrates the release profile of samples subjected to low speed and low temperature processes.

FIG. 19D is illustrative for the release rate per 9 mm catheter, line 1606a illustrates the minimum target therapeutic agent release profile. Line 1606b illustrates the maximum target therapeutic agent release profile. Line 1606c, interconnecting the inverted, triangle-shaped data points, illustrates the release profile of samples subjected to high speed and high temperature processes. Line 1606d, interconnecting the circle-shaped data points, illustrates the release profile of samples subjected to high speed and low temperature processes. Line 1606e, interconnecting the upright triangle-shaped data points, illustrates the release profile of samples subjected to low speed and high temperature processes. Line 1606f, interconnecting the diamond-shaped data points, illustrates the release profile of samples subjected to low speed and low temperature processes.

As shown in FIGS. 19A and 19B, the in vitro drug release profiles for the monofilament samples having a PCL-meloxicam coating with a ˜100-130 μm thickness achieved and exceeded the target release profile for the 6 mm and 9 mm catheters. The dip recorded in the cumulative release profiles at the 10-day time point may be due to a measurement error stemming from a high-performance liquid chromatographic method calibration error. The release profiles have an initial burst release followed by a sustained release over 14 days.

Referring additionally to FIGS. 19C and 19D, the plots of the release rates show the change in release rate over time with some plateau in the release around days 7-10. There was an initial burst release within the first hour, with a rate of ˜1.3 mg/d for a 6 mm catheter and ˜2.0 mg/d for a 9 mm catheter. After the initial burst, the release rates began to stabilize and remained relatively constant from day 2 through day 7, which were within the target daily release rate range of 0.0675-0.135 mg/d. The daily release rate then dropped slightly below the target range at day 14. For the 9 mm catheter, the release rates for day 2 through day 7 were slightly above the maximum target release rate of 0.135 mg/d, and then the daily release rate dropped to around the minimum target release rate of 0.0675 mg/d. The in vitro release results indicate that a 100-130 μm coating for 6 mm and 9 mm catheters are candidates for achieving the target release in vivo. Additionally, the effect of the processing conditions, including screw speed and processing temperature, is minimal in this example, indicating that the process is robust within the processing window tested.

### Micro-Injection Molding Example 8

In this example, a two-shot cylinder mold having dimensions (e.g., wall thickness and length) representative of a commercial catheter was designed, wherein the first shot was a polymer-only liner material for providing mechanical integrity and the second shot was a PCL-meloxicam coating jacket for controlling release of meloxicam to local subcutaneous tissue. The cylinder mold included a plurality of inserts for differing wall thicknesses of the first and second shots and two lengths corresponding with 6 mm and 9 mm catheters. The mold also had dual gating to facilitate balancing of outflow and pressure. The core pin dimension was held constant at 0.432 mm. The minimum wall thickness was 0.076 for each of the first shot and the second shot, resulting in a minimum overall wall thickness of 0.152 mm and an overall minimum outer diameter of 0.736 mm. The maximum overall wall thickness was 0.254 mm with an overall maximum outer diameter of 0.940 mm.

The two-shot cylinder molded part was formed using a 20-ton Sodick (Model LP20EH2, Serial 1073) molding machine. The material used for the first shot was high density polyethylene (“HDPE”) Borealis Bormed HE7541-PH. The material used for the second shot was PC17, further discussed above in Example 12, having 50 wt. % meloxicam loading, supplied as pellets formed by twin-screw extrusion in a pre-processing compounding step. For the first shot, HDPE parts were produced at three different wall thicknesses: 0.076 mm, 0.102 mm, and 0.127 mm, for each of the 6 mm and 9 mm catheter lengths.

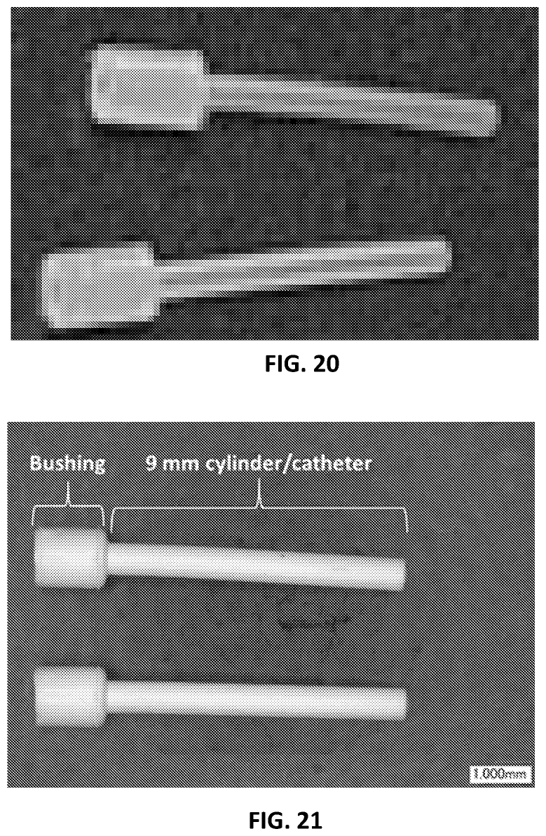

For the first shot of HDPE, the parts achieved complete fill for wall thicknesses down to 0.076 mm for both the 6 mm and 9 mm catheter lengths. All parts were easily removed from the core pin without use of a mold release. FIG. 20 shows an exemplary molded part for the first shot of HDPE for the 6 mm catheter length. For the second shots of PC17 having 50 wt. % meloxicam, complete fill was achieved for wall thicknesses of 0.127 mm, 0.152 mm, and 0.178 mm for the 9 mm length. FIG. 21 shows an exemplary molded for the first shot of HDPE and the second shot of PCL having 50 wt. % meloxicam loading for the 9 mm catheter length. Because the 9 mm length is more challenging to fill than the shorter 6 mm length, it was assumed that the complete fill for second shot wall thicknesses of 0.127 and 0.152 mm is achievable for the 6 mm length. The 0.178 mm wall thickness for the 6 mm catheter was tested and achieved complete fill.

Table 3 below shows the fill results for the second shot for the 6 mm and 9 mm catheter lengths, the meloxicam dose loaded in the coating, and the catheter dimensions achieved. The meloxicam dose loaded in each catheter part for the 6 mm length is within the range of 0.945-1.89 mg for the 14-day target dose and exceed this 14-day target dose range for the 9 mm length parts. The overall outer diameter for the two-shot molded catheters is 0.889-0.940 mm, which meets the guidance that the overall outer diameter of the catheter should not exceed 1 mm in this example.

TABLE 3
Fill results for the second shots with PC17 having 50
wt. % meloxicam for the 6 mm and 9 mm catheter lengths

| Length | 6 mm | 9 mm | 9 mm | 9 mm | 9 mm |
|---|---|---|---|---|---|
| 2^{nd} Shot Wall | 0.178 | 0.127 | 0.152 | 0.152 | 0.178 |
| Thickness (mm) | | | | | |
| 2^{nd} Shot Fill | Com- | Com- | Com- | Com- | Com- |
| | plete | plete | plete | plete | plete |
| Meloxicam dose | 1.710 | 1.832 | 2.125 | 2.272 | 2.565 |
| loaded (mg) | | | | | |
| 1^{st} Shot Wall | 0.076 | 0.102 | 0.076 | 0.102 | 0.076 |
| Thickness (mm) | | | | | |
| Overall Outer | 0.940 | 0.889 | 0.889 | 0.940 | 0.940 |
| Diameter (mm) | | | | | |

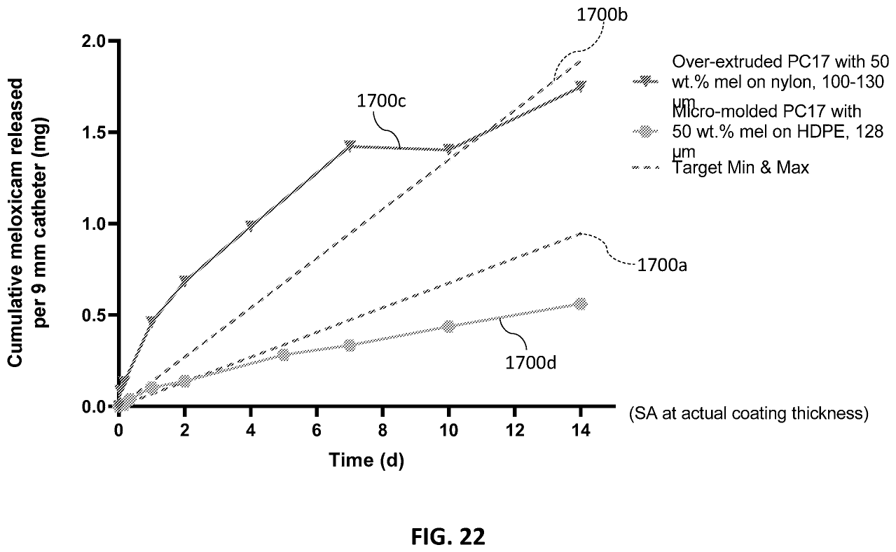

Referring to FIG. 22, the in vitro cumulative meloxicam release is presented per 9 mm catheter. Line 1700a illustrates the minimum target therapeutic agent release profile. Line 1700b illustrates the maximum target therapeutic agent release profile. Line 1700c illustrates the therapeutic agent release profile for an over-extruded PC17 coating having 50 wt. % meloxicam loading on nylon filaments, wherein the coating measures ˜100-130 μm. Line 1700d illustrates the therapeutic agent release profile for a micro-molded cylinder part with a second shot of PC17 having 50 wt. % meloxicam loading over a micro-molded first shot of HDPE, the micro-molded second shot having a thickness of 128 μm.

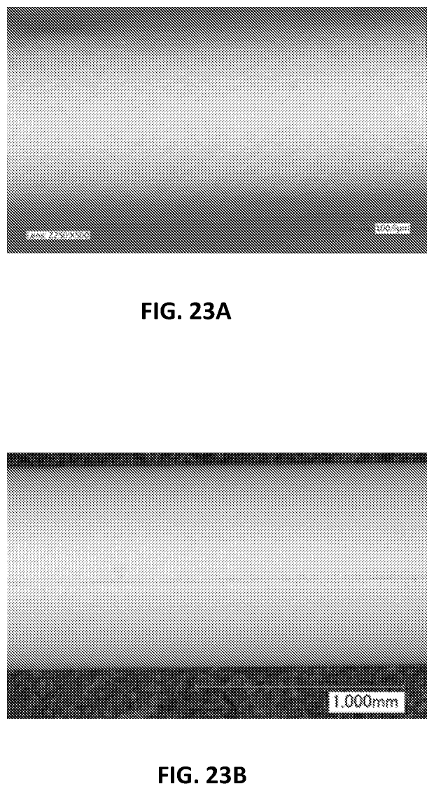

The release from the micro-molded parts (1700d) approached the minimum target release as compared to the over-extruded samples which approached the maximum target release (1700c). The release profile of the micro-molded parts (1700d) shows sustained release over 14 days with a relatively linear release curve. Upon further characterization of the two samples, a difference in surface texture was observed, which impacts the drug release. The over-extruded samples, as shown in FIG. 23A, at all four conditions tested as discussed above, have a semi-rough surface texture, while the micro-molded samples, as shown in FIG. 23B have a smoother surface finish.

### Coating Thickness Example 9

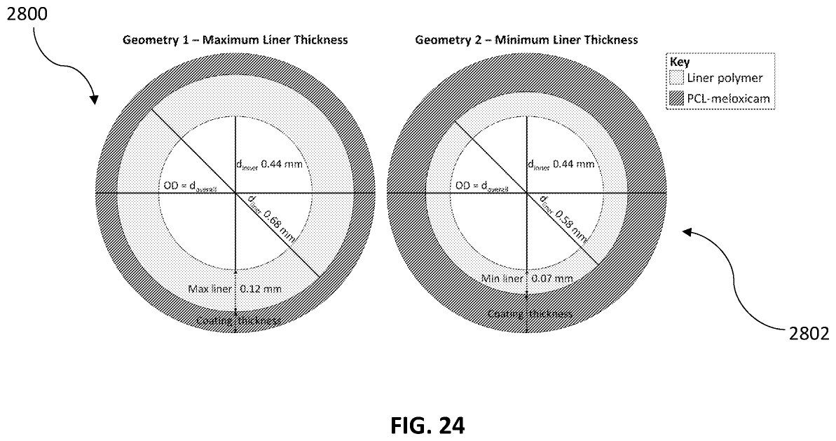

For EVA-meloxicam formulations and PCL-meloxicam formulations discussed above, coating thicknesses were calculated from the experimental results for different formulations. Referring to FIG. 24, the thicknesses were calculated for a maximum liner thickness geometry 2800 and minimum liner thickness geometry 2802 according to minimum target release rate (0.0675 mg/day, 0.945 mg total of meloxicam at 14 days), maximum target release rate (0.135 mg/day, 1.89 mg total of meloxicam at 14 days), and three different catheter lengths.

The average release rate normalized to surface area was calculated from the in vitro elution results provided above. Specifically, the average release rate was calculated as the weighted average of the release rate over 14 days, since the release rate was not constant over the 14-day period. The average release rate was normalized to the surface area of the sample, depending on the catheter length. The following equations were used to calculate the coating thicknesses based on the experimental average release rates normalized to surface area, wherein m=mass, V=volume, p=density, w=weight fraction, A=area, r=radius, d=diameter, h=height, and ϕ=volume fraction. These calculations assume that the catheter is a hollow cylinder.

### The Equation for the Target Surface Area is as Follows:

$${A}_{target} = \frac{Target release rate}{Avg experimental release rate normalized to SA} [ = ] \frac{mg}{d} \times \frac{d * {mm}^{2}}{mg} [ = ] {mm}^{2}$$

The equation for the overall radius of the catheter is as follows, wherein the target surface area and height are known:

$${A}_{cylinder} = 2 π rh + 2 π {r}^{2}$$

Assuming there are no ends for the surface area of the catheter, the surface area may be simplified to:

A=2πrh

### The Equation for the Overall Diameter is as Follows:

dₒᵥₑᵣₐₗₗ=2rₒᵥₑᵣₐₗₗ

### The Equation for the Coating Thickness is as Follows:

$$Thickness = {r}_{overall} - {r}_{liner}$$

In order to determine if the coating thickness of the polymer-meloxicam layer has a sufficient amount of meloxicam to achieve the target meloxicam 14-day dose (0.945-1.89 mg), the mass of the meloxicam loaded in the coating was calculated based on the coating volume using the following equations. The equation for the coating volume is:

$${V}_{coating} = {V}_{tube} = π h [ {( {d}_{overall} / 2 )}^{2} - {( {d}_{l i n e r} / 2 )}^{2} ] [ = ] {mm}^{3}$$

The equation for the mass of meloxicam loaded, based on the coating volume calculated above, is as follows, wherein the volume of the coating, density of the meloxicam, density of the polymer, and weight of the drug are known:

$$m = ρ V \to V = \frac{m}{ρ}$$

The equations for mass balance are as follows:

$${m}_{coating} = {m}_{d r u g} + {m}_{polymer}$$

$${m}_{t o t a l} = \frac{{m}_{d r u g}}{{w}_{d r u g}}$$

$${m}_{polymer} = {m}_{t o t a l} - {m}_{d r u g} = \frac{{m}_{d r u g}}{{w}_{d r u g}} - {m}_{d r u g} = {m}_{d r u g} ( \frac{1}{{w}_{d r u g}} - 1 )$$

The equations for volume balance are as follows:

$${V}_{coating} = {V}_{drug} + {V}_{polymer}$$

$${V}_{coating} = \frac{{m}_{d r u g}}{{ρ}_{d r u g}} + \frac{{m}_{polymer}}{{ρ}_{polymer}}$$

When substituting in the polymer mass, the equation becomes:

$${V}_{c oating} = \frac{{m}_{drug}}{{ρ}_{drug}} + \frac{{m}_{drug} ( \frac{1}{{w}_{drug}} - 1 )}{{ρ}_{polymer}} = {m}_{drug} ( \frac{1}{{ρ}_{drug}} + \frac{( \frac{1}{{w}_{drug}} - 1 )}{{ρ}_{polymer}} )$$

To solve for the meloxicam mass, the following equation was used:

$${m}_{d r u g} = \frac{{V}_{c o a t i n g}}{( \frac{1}{{ρ}_{d r u g}} + \frac{( \frac{1}{{w}_{d r u g}} - 1 )}{{ρ}_{polymer}} )} [ = ] \frac{{mm}^{3}}{\frac{1}{mg / {mm}^{3}}} [ = ] mg$$

If the mass of meloxicam was greater than or equal to the target 14-day meloxicam dose, then the coating thickness calculated based on the experimental average release rates, normalized to surface area, was reported. However, if the mass of meloxicam was below the target 14-day meloxicam dose, then the coating thickness was calculated based on the target meloxicam dose. In this case, the coating thickness based on the target meloxicam dose is greater than the coating thickness based on the experimental release rates. The coating thickness based on the target meloxicam dose was calculated according to the following equation:

$${V}_{c o a t i n g} = {m}_{d r u g ( t a r g e t )} ( \frac{1}{{ρ}_{d r u g}} + \frac{( \frac{1}{{w}_{d r u g}} - 1 )}{{ρ}_{polymer}} ) [ = ] {mm}^{3}$$

### To Calculate the Overall Outer Diameter, Wherein the Coating Volume and Liner Diameter is Known:

$${V}_{c o a t i n g} = π h [ {( {d}_{overall} / 2 )}^{2} - {( {d}_{liner} / 2 )}^{2} ]$$

### To Solve for the Overall Outer Diameter:

$${d}_{overall} = \sqrt{\frac{{π ( {d}_{l i n e r} )}^{2} h + 4 V}{π h}}$$

### To Calculate the Overall Radius:

dₒᵥₑᵣₐₗₗ=2rₒᵥₑᵣₐₗₗ

### To Solve for the Coating Thickness:

$$Thickness = {r}_{overall} - {r}_{liner}$$

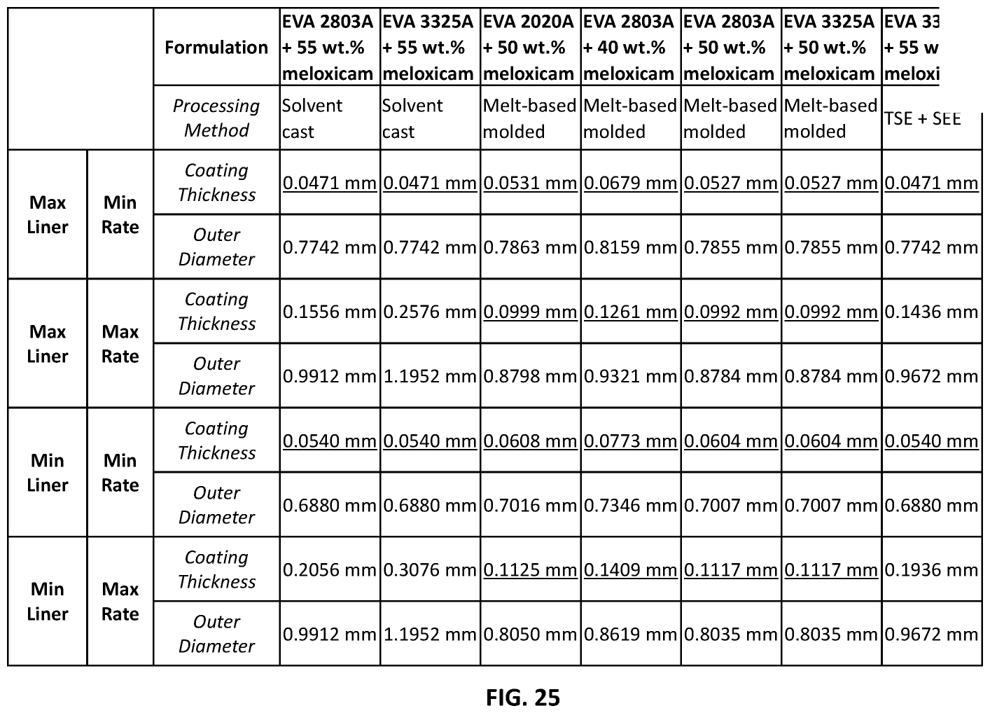

FIGS. 25-27 provide the thickness of the tested EVA-meloxicam coatings for 13 mm, 9 mm, and 6 mm catheters, respectively and corresponding overall catheter outer diameter for the minimum and maximum target release rates for each of the maximum liner thickness and the minimum liner thickness. The coating thicknesses were calculated based on the experimental average release rates, unless the coating thickness value is underlined, indicating that the coating thickness was calculated based on the target meloxicam dose.

Referring to FIG. 25, corresponding with a 13 mm catheter length, assuming an ideal overall catheter diameter of less than 1 mm and an operational overall catheter diameter of 1.5 mm or less, the EVA-meloxicam formulations EVA 2803A having 40-55 wt. % meloxicam loading subject to all processing methods tested met the ideal overall diameter. Additionally, the EVA-meloxicam formulation EVA 2020A having 50 wt. % meloxicam loading met the ideal overall diameter. The EVA-meloxicam formulation EVA 3325A having 50-55 wt. % meloxicam loading subject to melt-based molding and extrusion processing methods met the ideal overall diameter. The EVA-meloxicam formulation EVA 3325A having 55 wt. % meloxicam loading subject to solvent-based method met the ideal overall diameter for each of the maximum and minimum liner diameters meeting the minimum release rate; however, this formulation met an operational overall diameter for each of the maximum and minimum liner diameters meeting the maximum release rate.

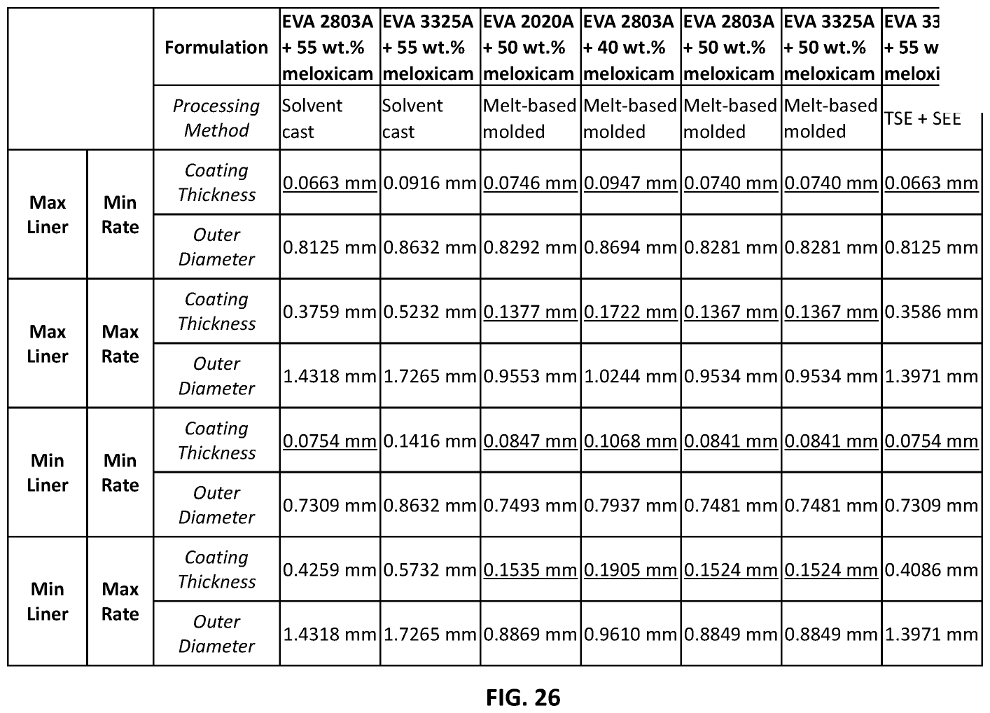

Referring to FIG. 26, corresponding with a 9 mm catheter length and assuming the same catheter diameter categories above, the EVA-meloxicam formulations EVA 2803A having 40-55 wt. % meloxicam loading subject to all processing methods tested met the ideal overall diameter for each of the maximum and minimum liner diameters meeting the minimum release rate; however, these formations met an operational overall diameter for each of the maximum and minimum liner diameters meeting the maximum release rate, with the exception of EVA 2803A having 50 wt. % meloxicam subject to melt-based molding that met the ideal overall diameter for all configurations. The EVA-meloxicam formulations EVA 2020A having 50 wt. % meloxicam loading EVA 3325A having 50 wt. % meloxicam loading subject to melt-based molding processing method met the ideal overall diameter. The EVA-meloxicam formulations EVA 3325A having 55 wt. % meloxicam loading subject to the solvent-based and extrusion processing methods met the ideal overall diameter for each of the maximum and minimum liner diameters meeting the minimum release rate; however, these formulations met an operational overall diameter for each of the maximum and minimum liner diameters meeting the maximum release rate.

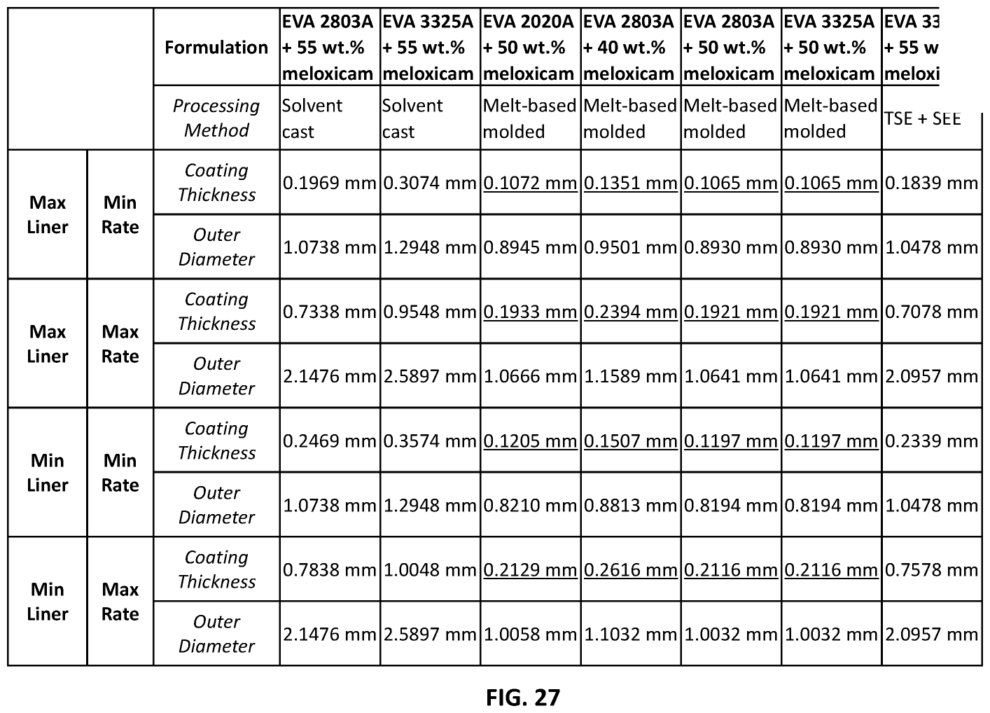

Referring to FIG. 27, corresponding with a 6 mm catheter length and assuming the same catheter diameter categories above, the EVA-meloxicam formulations EVA 2803A having 40-50 wt. % meloxicam loading subject to melt-based molding processing method, EVA 2029A having 50 wt. % loading meloxicam subject to melt-based molding processing method, and EVA 3325A having 55 wt. % meloxicam subject to melt-based molding processing method met the ideal overall diameter for each of the maximum and minimum liner diameters meeting the minimum release rate; however, these formulations met an operational overall diameter for each of the maximum and minimum liner diameters meeting the maximum release rate. The EVA-meloxicam formulations EVA 2803A having 55 wt. % meloxicam loading subject to solvent-based processing method and EVA 3325A having 55 wt. % meloxicam loading subject to solvent-based and extrusion processing methods met the ideal overall diameter for each of the maximum and minimum liner diameters meeting the minimum release rate.

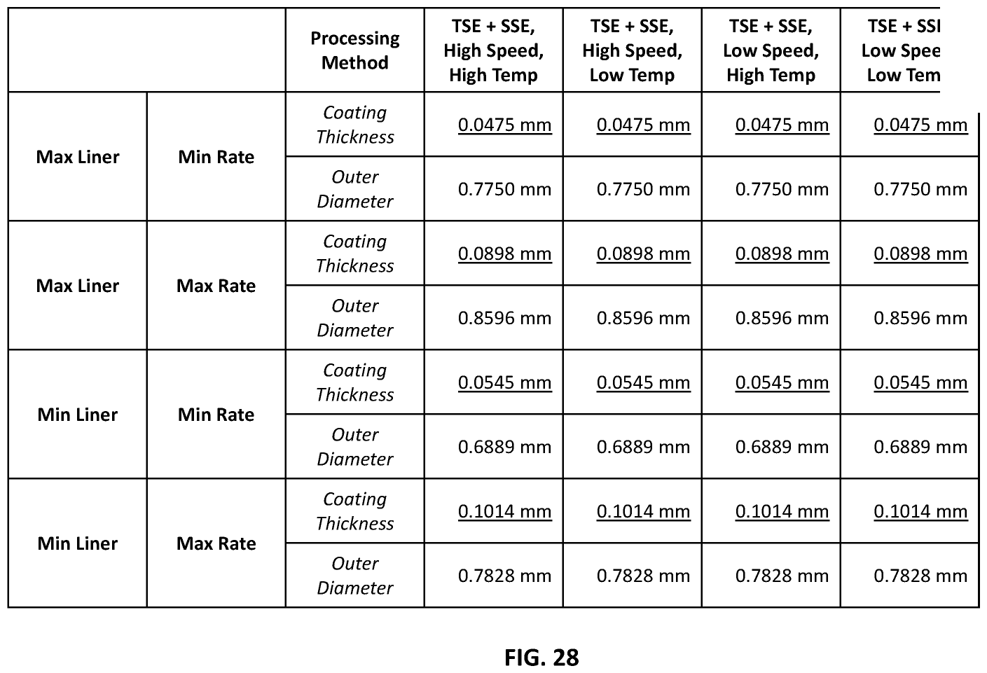

FIGS. 28-30 provide the thickness of the tested PCL-meloxicam coatings for 13 mm, 9 mm, and 6 mm catheters, respectively and corresponding overall catheter outer diameter for the minimum and maximum target release rates for each of the maximum liner thickness and the minimum liner thickness. The coating thicknesses were calculated based on the experimental average release rates, unless the coating thickness value is underlined, indicating that the coating thickness was calculated based on the target meloxicam dose. All calculations were performed according to the PC17 with 50 wt. % meloxicam loading formulations discussed above, according to different processing methods.

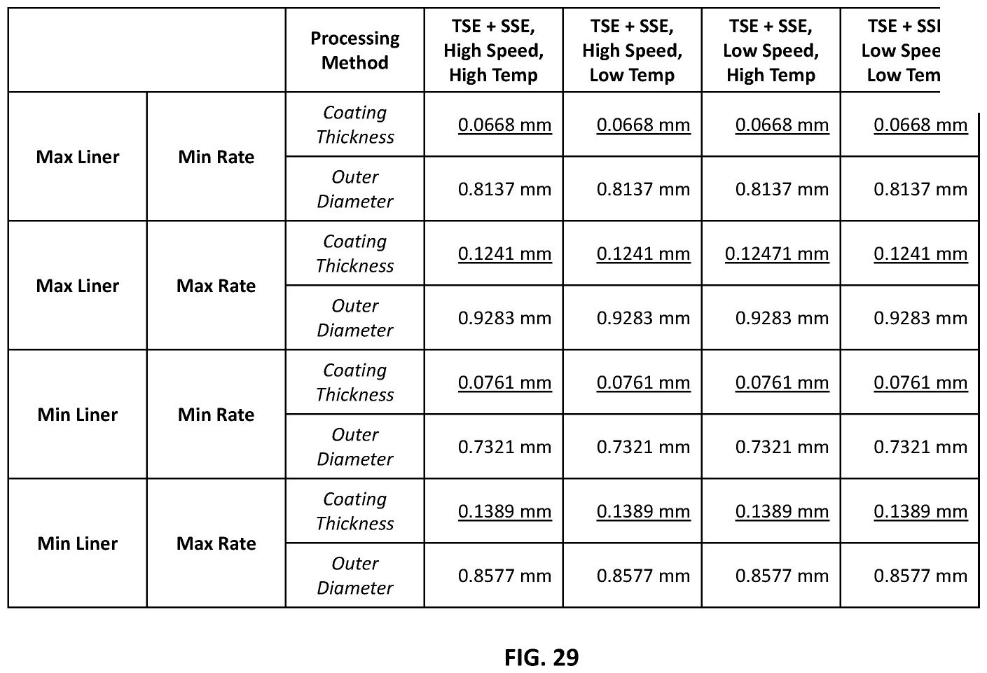

Referring to FIG. 28, corresponding with a 13 mm catheter length, assuming an ideal overall catheter diameter of less than 1 mm and an operational overall catheter diameter of 1.5 mm or less, the PCL-meloxicam formulations subject to all processing methods tested met the ideal overall diameter. Referring also to FIG. 29, corresponding with a 9 mm catheter length and assuming the same catheter diameter categories above, the PCL-meloxicam formulations subject to all processing methods tested met the ideal overall diameter.

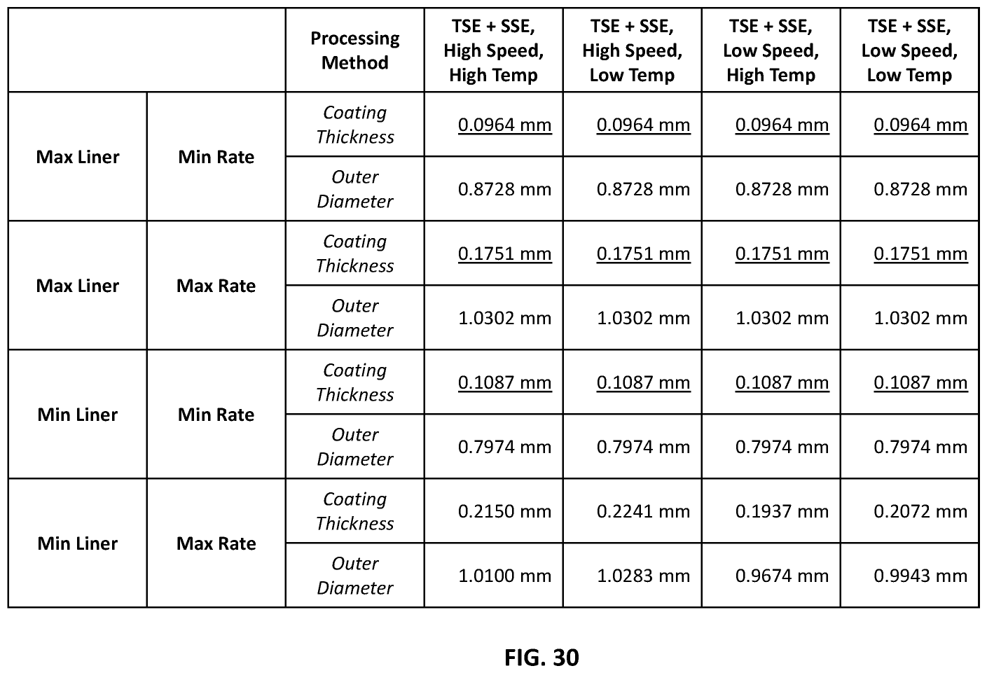

Referring to FIG. 30, corresponding with a 6 mm catheter length and assuming the same catheter diameter categories above, the PCL-meloxicam formulations subject to a low speed processing method met the ideal overall diameter for each of the maximum and minimum liner diameters meeting the minimum release rate and for the minimum liner diameter meeting the maximum release rate; however, these formulations met the operational overall diameter for the maximum liner diameter meeting the maximum release rate. The PCL-meloxicam formulations subject to a high speed processing method met the ideal overall diameter for each of the maximum and minimum liner diameters meeting the minimum release rate; however, these formulations met the operational overall diameter for each of the maximum and minimum liner diameters meeting the maximum release rate.

Tables 4 and 5 below provide the exemplary minimum wall thicknesses for each of the two tested polymer matrices at 6 mm, 9 mm, and 13 mm catheter lengths. Each of the provided measurements assumes a minimum target meloxicam dose of 0.945 mg, 100% release over a 14-day period, and a meloxicam loading of 55 wt. %. However, the measurements do not take into consideration the impact of surface area on release rate.

TABLE 4
Minimum coating thicknesses for EVA and PCL formulations normalized
to 6 mm, 9 mm, and 13 mm catheters for maximum liner thickness.
Maximum Liner Thickness (d = 0.68 mm)

| | Polymer | | | | | |
|---|---|---|---|---|---|---|
| | EVA | | | PCL | | |
| | Catheter (mm) | | | | | |
| | 6 | 9 | 13 | 6 | 9 | 13 |
| Overall | 0.871 | 0.713 | 0.774 | 0.854 | 0.801 | 0.765 |
| Diameter | | | | | | |
| (mm) | | | | | | |
| Coating | 0.0956 | 0.0663 | 0.0471 | 0.0872 | 0.0603 | 0.0427 |
| Thickness | | | | | | |
| (mm) | | | | | | |
| Coating | 95.6 | 66.3 | 47.1 | 87.2 | 60.3 | 42.7 |
| Thickness | | | | | | |
| (μm) | | | | | | |

TABLE 5
Minimum coating thicknesses for EVA and PCL formulations normalized
to 6 mm, 9 mm, and 13 mm catheters for minimum liner thickness.
Minimum Liner Thickness (d = 0.58 mm)

| | Polymer | | | | | |
|---|---|---|---|---|---|---|
| | EVA | | | PCL | | |
| | Catheter (mm) | | | | | |
| | 6 | 9 | 13 | 6 | 9 | 13 |
| Overall | 0.796 | 0.731 | 0.688 | 0.777 | 0.718 | 0.678 |
| Diameter | | | | | | |
| (mm) | | | | | | |
| Coating | 0.1078 | 0.0754 | 0.0540 | 0.0986 | 0.0688 | 0.0491 |
| Thickness | | | | | | |
| (mm) | | | | | | |
| Coating | 107.8 | 75.4 | 54.0 | 98.6 | 68.8 | 49.1 |
| Thickness | | | | | | |
| (μm) | | | | | | |

While embodiments of the invention have been described as having exemplary designs, the embodiments of the present disclosure may be further modified within the spirit and scope of this disclosure. This application is therefore intended to cover any variations, uses, or adaptations of the disclosed embodiments using its general principles.

What is claimed is:

1. An infusion device comprising:
a base;
an adhesive configured to couple the base to a skin of a patient; and
a catheter having a therapeutic coating, the coating comprising a polymer matrix and a therapeutic agent, the coating defining a thickness of at least 40 μm and a therapeutic agent loading of at least 30 wt. %.

2. The infusion device of claim 1, wherein the therapeutic agent comprises a nonsteroidal anti-inflammatory drug.

3. The infusion device of claim 2, wherein the therapeutic agent comprises meloxicam.

4. The infusion device of claim 1, wherein the polymer matrix comprises at least one of ethyl vinyl acetate, thermoplastic polyurethane, or poly(caprolactone).

5. The infusion device of claim 4, wherein the polymer matrix is ethyl vinyl acetate and has a melt index of about 33 dg/min. and a vinyl acetate content of about 25%.

6. The infusion device of claim 1, wherein the catheter is configured to remain inserted in the patient for at least 7 days, and wherein the catheter is configured to continuously release the therapeutic agent while inserted.

7. The infusion device of claim 1, wherein the therapeutic agent is comprised of a plurality of discrete particles embedded in the polymer matrix.

8. The infusion device of claim 1, wherein the therapeutic coating comprises at least 40 wt. % of the therapeutic agent.

9. The infusion device of claim 1, wherein the therapeutic coating further comprises a second therapeutic agent.

10. An extended release coating comprising a polymer matrix having a therapeutic agent comprising at least 45 wt. % of the coating, the coating defining a thickness of at least 40 μm.

11. The coating of claim 10, wherein the therapeutic agent is a nonsteroidal anti-inflammatory drug.

12. The coating of claim 11, wherein the therapeutic agent is meloxicam.

13. The coating of claim 10, wherein the polymer matrix is at least one of ethyl vinyl acetate, thermoplastic polyurethane, or poly(caprolactone).

14. The coating of claim 10, wherein the therapeutic agent is comprised of a plurality of discrete particles embedded in the polymer matrix.

15. The coating of claim 14, wherein the plurality of discrete particles has a particle size of at least d90=20 μm.

16. The coating of claim 10, wherein the coating is configured to release from approximately 0.75 mg of the therapeutic agent to approximately 1.89 mg of the therapeutic agent over at least 7 days.

17. A method for applying a therapeutic coating to a catheter, the method comprising:
compounding a mixture of a polymer and a therapeutic agent to form a polymer matrix and therapeutic agent mixture with a therapeutic agent loading of at least 30 wt. %; and
shaping the mixture to form a coating for application to a catheter, wherein the coating defines a thickness of at least 40 μm.

18. The method of claim 16, wherein the mixture is applied to the catheter by overextrusion using a single screw extruder.

19. The method of claim 16, wherein a polymer is injected into a mold to form a liner and the mixture is injected into the mold over the liner to form the coating.

20. The method of claim 16, wherein the coating defines a thickness of at least 50 μm.
